SEQUENCE LISTING (15 sequences)

SEQ ID NO: 1 - protein, 1255 aa, unidentified
  source 1..1255: note = SARS-CoV-1 Spike Protein
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - protein, 222 aa, unidentified
  source 1..222: note = SARS-CoV-1 Spike Protein receptor binding domain
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 3 - protein, 76 aa, unidentified
  source 1..76: note = SARS-CoV-1 Envelope Protein
MYSFVSEETGTLIVNSVLLFLAFVVFLLVTLAILTALRLCAYCCNIVNVSLVKPTVYVYSRVKNLNSSEGVPDLLV

SEQ ID NO: 4 - protein, 221 aa, unidentified
  source 1..221: note = SARS-CoV-1 Membrane Protein
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 5 - protein, 422 aa, unidentified
  source 1..422: note = SARS-CoV-1 Nucleocapsid Protein
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 6 - protein, 1273 aa, unidentified
  source 1..1273: note = SARS-CoV-2 Spike Protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 223 aa, unidentified
  source 1..223: note = SARS-CoV-2 Spike Protein receptor binding domain
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 8 - protein, 75 aa, unidentified
  source 1..75: note = SARS-CoV-2 Envelope Protein
MYSFVSEETGTLIVNSVLLFLAFVVFLLVTLAILTALRLCAYCCNIVNVSLVKPSFYVYSRVKNLNSSRVPDLLV

SEQ ID NO: 9 - protein, 222 aa, unidentified
  source 1..222: note = SARS-CoV-2 Membrane Protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 10 - protein, 419 aa, unidentified
  source 1..419: note = SARS-CoV-2 Nucleocapsid Protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 11 - protein, 1353 aa, unidentified
  source 1..1353: note = MERS-CoV Spike protein
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSTVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 12 - protein, 122 aa, unidentified
  source 1..122: note = MERS-CoV Spike protein receptor binding domain
ECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKC

SEQ ID NO: 13 - protein, 82 aa, unidentified
  source 1..82: note = MERS-CoV Envelope protein
MLPFVQERIGLFIVNFFIFTVVCAITLLVCMAFLTATRLCVQCMTGFNTLLVQPALYLYNTGRSVYVKFQDSKPPLPPDEWV

SEQ ID NO: 14 - protein, 219 aa, unidentified
  source 1..219: note = MERS-CoV Membrane protein
MSNMTQLTEAQIIAIIKDWNFAWSLIFLLITIVLQYGYPSRSMTVYVFKMFVLWLLWPSSMALSIFSAVYPIDLASQIISGIVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 15 - protein, 413 aa, unidentified
  source 1..413: note = MERS-CoV Nucleocapsid protein
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

(19) United States
(12) Patent Application Publication
(10) Pub. No.: US 2025/0041278 A1
(43) Pub. Date: Feb. 6, 2025

(54) TLR8 AGONIST FOR MODULATING IMMUNE RESPONSE

(71) Applicant: The Children's Medical Center Corporation, Boston, MA (US)

(72) Inventors: David J. Dowling, Milton, MA (US); Ofer Levy, Cambridge, MA (US); Simon D. van Haren, Wayland, MA (US); Katherine Chew, Cambridge, MA (US); Dheeraj Soni, Brighton, MA (US); Maria De Leon, Cambridge, MA (US)

(73) Assignee: The Children's Medical Center Corporation, Boston, MA (US)

(21) Appl. No.: 18/696,547

(22) PCT Filed: Sep. 29, 2022
(86) PCT No.: PCT/US2022/077234

§ 371 Date: Mar. 28, 2024

Related U.S. Application Data
(60) Provisional application No. 63/250,940, filed on Sep. 30, 2021.

(51) Int. Cl. A61K 31/4184; A61K 39/00; A61K 39/39; A61P 31/14; A61P 37/04

(52) U.S. Cl. CPC A61K 31/4184; A61K 39/39; A61P 31/14; A61P 37/04; A61K 2039/55511; A61K 2039/55572

(57) ABSTRACT

Provided herein are uses for an immunostimulatory compound for stimulating an immune response when administered either alone or as an adjuvant in a vaccine. Also provided herein are kits, compositions, and methods of administration for the compound described for proliferative disease, inflammatory disease, autoimmune disease, infectious disease, or chronic disease, in a subject in need thereof. Using the compound as a vaccine adjuvant enables effective immunization in vulnerable populations.

### RELATED APPLICATIONS

This application claims the benefit under 35 U.S.C. § 119(e) to U.S. Provisional Application No. 63/250,940 filed on Sep. 30, 2021 entitled “TLR8 AGONIST FOR MODULATING IMMUNE RESPONSE,” the entire contents of which are incorporated herein by reference.

### FEDERALLY SPONSORED RESEARCH

This invention was made with government support under contract number 75N93019C00044 awarded by the National Institutes of Health. The government has certain rights in the invention.

### REFERENCE TO AN ELECTRONIC SEQUENCE LISTING

The contents of the electronic sequence listing (C123370221WO00-SEQ-RE.xml; Size: 23,330 bytes; and Date of Creation: Sep. 28, 2022) is herein incorporated by reference in its entirety.

### BACKGROUND

The immune system is crucial to the overall health of many species including humans, playing key roles not only in the defense against infectious diseases and cancers, but also in the development of allergies and autoimmune diseases. Certain small molecules can modulate the immune system by acting as activators or inhibitors. These compounds are of particular interest as they may be administered to individuals to either treat or protect against disease.

### SUMMARY

Provided herein is a compound for use in enhancing an immune response in a subject, such as a human. Therapeutic and/or prophylactic uses of the compound are described. In some embodiments, the compound is used alone to stimulate immunity in a subject. In some embodiments, the compound is used as an adjuvant in compositions including vaccines for diseases, e.g., proliferative disease, inflammatory disease, autoimmune disease, infectious disease, or chronic disease, in a subject in need thereof. Adjuvants can enhance, prolong, and modulate immune responses to vaccinal antigens to maximize protective immunity. In some embodiments, using the compound as a vaccine adjuvant enables effective immunization in vulnerable populations (e.g., neonates, the elderly, or immunocompromised individuals). In some embodiments, the compound enhances both innate and adaptive immune responses.

Accordingly, some aspects of the present disclosure provide methods of enhancing an immune response in a subject in need thereof, the method comprising administering to the subject an effective amount of a compound of Formula (I). Also provided are methods of treating a disease or reducing the risk of a disease, the method comprising administering to a subject in need thereof an effective amount of a compound of Formula (I). The compound of Formula (I) is as follows:

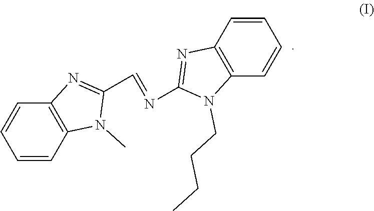 [SMILES: CCCCn1c(/N=C/c2nc3ccccc3n2C)nc2ccccc21]

In some embodiments, the immune response is an innate immune response. In some embodiments, the immune response is an adaptive immune response. In some embodiments, the immune response is a cell-mediated immune response. In some embodiments, the immune response is a heterologous immune response.

In some embodiments, the compound is adsorbed onto alum. In some embodiments, the compound is lipidated. In some embodiments, the compound is in an aqueous formulation. In some embodiments, the compound is in an emulsion formulation. In some embodiments, the compound is in a nanoparticle formulation.

In some embodiments, the compound is administered to the subject more than once.

In some embodiments, the subject has or is at risk of developing an infectious disease. In some embodiments, the infectious disease is caused a bacterium, a mycobacterium, a fungus, a virus, a parasite, or a prion. In some embodiments, the infectious disease is sepsis. In some embodiments, the subject has or is at risk of developing cancer. In some embodiments, the cancer is metastatic cancer. In some embodiments, the cancer is a hematological cancer, lung cancer, breast cancer, brain cancer, gastrointestinal cancer, liver cancer, kidney cancer, bladder cancer, pancreatic cancer, ovarian cancer, testicular cancer, prostate cancer, endometrial cancer, muscle cancer, bone cancer, neuroendocrine cancer, connective tissue cancer, head and neck cancer, or skin cancer. In some embodiments, the subject has or is at risk of developing allergy. In some embodiments, the subject has radiation injury. In some embodiments, the subject is immune-senescent, immune-compromised, is infected with human immunodeficiency virus (HIV), has chronic lung disease, asthma, cardiovascular disease, cancer, a metabolic disorder, chronic kidney disease, liver disease, is malnourished, or is frail.

In some embodiments, the administration is systemic or local. In some embodiments, the administration is intramuscular, intradermal, oral, intravenous, topical, intranasal, intravaginal, or sublingual. In some embodiments, the administration is prophylactic.

In some embodiments, the subject is a human neonate, an infant, an adult, or an elderly individual. In some embodiments, the subject is a human adult. In some embodiments, the subject is an elderly individual. In some embodiments, the administration occurs when the subject is more than 65 years of age.

In some embodiments, the subject is a companion animal or a research animal. In some embodiments, the subject is an adult or elderly companion animal.

In some embodiments, the compound activates peripheral blood mononuclear cells (PBMCs). In some embodiments, the PBMCs are macrophages or dendritic cells. In some embodiments, the compound induces a T helper type 1 (Th1) response. In some embodiments, the compound induces a signaling molecule in the subject. In some embodiments, the signaling molecule is a proinflammatory or Th-polarizing cytokine or chemokine. In some embodiments, the signaling molecule is a cytokine selected from TNF, IL-6, IL-10, IL-12, GM-CSF, IFN-γ, IL1-β, and/or IP-10, a chemokine selected from CCL2 (MCP1), CCL5, and/or CXCL8 (IL-8), or a combination thereof.

In some embodiments, the compound enhances humoral immunity. In some embodiments, the compound induces the production of an immunoglobulin in the subject. In some embodiments, the immunoglobulin is an immunoglobulin G (IgG), an immunoglobulin A (IgA), or an immunoglobulin M (IgM). In some embodiments, the IgG is a subclass 1 IgG (IgG1) or a subclass 2 IgG (IgG2).

Further provided herein are compositions comprising an antigen and the compound of Formula (I). In some embodiments, the antigen comprises a protein or polypeptide. In some embodiments, the antigen comprises a nucleic acid encoding a protein or a polypeptide. In some embodiments, the nucleic acid is DNA or RNA. In some embodiments, the antigen is from a microbial pathogen. In some embodiments, the microbial pathogen is a bacterium, mycobacterium, fungus, virus, parasite, or prion.

In some embodiments, the bacterium is Bacillus anthracis, Bordetella pertussis, Corynebacterium diphtheriae, Clostridium tetani, Haemophilus influenzae type b, pneumococcus, Staphylococci spp., Streptococcus spp., Mycobacterium spp., Neiserria spp., Salmonella typhi, Vibrio cholerae, or Yersinia pestis.

In some embodiments, the virus is adenovirus, coronavirus, enterovirus such as polio virus, dengue virus, Ebola virus, herpes viruses such as herpes simplex virus, cytomegalovirus and varicella-zoster, measles, mumps, rubella, hepatitis A virus, hepatitis B virus, hepatitis C virus, human papilloma virus, Influenza virus, rabies, Japanese encephalitis, rotavirus, human immunodeficiency virus (HIV), respiratory syncytial virus (RSV), smallpox, yellow fever, dengue virus, or Zika virus.

In some embodiments, the virus is Middle East Respiratory Syndrome coronavirus (MERS-CoV), Severe Acute Respiratory Syndrome (SARS)-associated coronavirus (SARS-CoV)-1, or SARS-CoV-2. In some embodiments, the antigen is a MERS-CoV spike protein, a SARS-CoV-1 spike protein, or a SARS-CoV-2 spike protein. In some embodiments, the antigen is a MERS-CoV spike protein receptor binding domain (RBD), a SARS-CoV-1 spike protein RBD, or a SARS-CoV-2 spike protein RBD. In some embodiments, the antigen is a nucleic acid encoding a MERS-CoV spike protein, a MERS-CoV spike protein RBD, a SARS-CoV-1 spike protein, a SARS-CoV-1 spike protein RBD, a SARS-CoV-2 spike protein, or a SARS-CoV-2 spike protein RBD. In some embodiments, the antigen comprises a viral particle of MERS-CoV, SARS-CoV-1, or SARS-CoV-2. In some embodiments, the antigen comprises killed or inactivated MERS-CoV, SARS-CoV-1, or SARS-CoV-2. In some embodiments, the antigen comprises live attenuated MERS-CoV, SARS-CoV-1, or SARS-CoV-2.

In some embodiments, the parasite is Plasmodium spp., Leishmania, or a helminth.

In some embodiments, the fungus is Candida spp., Aspergillus spp., Cryptococcus spp., Mucormycete, Blastomyces dermatitidis, Histoplasma capsulatum, or Sporothrix schenckii.

In some embodiments, the antigen is a cancer-specific antigen. In some embodiments, the antigen is a heteroclitic epitope or a cryptic epitope derived from the cancer-specific antigen. In some embodiments, the cancer-specific antigen is a neoantigen. In some embodiments, the antigen comprises a lipopolysaccharide (LPS).

In some embodiments, the compound is conjugated to the antigen. In some embodiments, the compound is not conjugated to the antigen.

In some embodiments, the composition further comprises a pharmaceutically acceptable carrier. In some embodiments, the composition is a vaccine composition.

In some embodiments, the compound is an adjuvant. In some embodiments, the antigen is adsorbed onto alum. In some embodiments, the compound is adsorbed onto alum. In some embodiments, the vaccine composition further comprises a second adjuvant. In some embodiments, the second adjuvant is an agonist of a Pattern Recognition Receptor (PRR). In some embodiments, the PRR is selected from the group consisting of Toll-like receptors (TLRs), NOD-like receptors (NLRs), RIG-I-like receptor (RLR), C-type Lectin receptors (CLRs), and a stimulator of interferon genes (STING). In some embodiments, second adjuvant is bound to or adsorbed to alum. In some embodiments, the second adjuvant is alum. In some embodiments, the second adjuvant is an emulsion. In some embodiments, the vaccine composition is a subunit vaccine, an attenuated vaccine, or a conjugate vaccine.

Also provided herein are methods of enhancing an immune response to an antigen in a subject in need thereof, the method comprising administering to the subject an effective amount of a composition comprising an antigen and an effective amount of the compound of Formula (I).

In some embodiments, the subject is a human neonate, an infant, an adult, or an elderly individual. In some embodiments, the subject is a human adult. In some embodiments, the subject is an elderly human. In some embodiments, the administration occurs when the subject is more than 65 years of age. In some embodiments, the subject is immune-senescent, immune-compromised, is infected with human immunodeficiency virus (HIV), has chronic lung disease, asthma, cardiovascular disease, cancer, a metabolic disorder, chronic kidney disease, liver disease, is malnourished, or is frail. In some embodiments, the subject is infected with SARS-CoV-2. In some embodiments, the subject is at risk for SARS-CoV-2.

In some embodiments, the composition induces a cell-mediated immune response. In some embodiments, the composition induces a heterologous immune response. In some embodiments, the composition induces a T helper type 1 (Th1) immune response.

In some embodiments, the composition induces the production of a signaling molecule in the subject. In some embodiments, the signaling molecule is selected from a proinflammatory or Th-polarizing cytokine or chemokine. In some embodiments, the signaling molecule is a cytokine selected from TNF, IL-6, IL-10, IL-12, GM-CSF, IFN-γ, IL1-0, and/or IP-10, a chemokine selected from CCL2 (MCP1), CCL5, and/or CXCL8 (IL-8), or a combination thereof.

In some embodiments, the composition induces the production of an immunoglobulin in the subject. In some embodiments, the immunoglobulin is specific to the antigen. In some embodiments, the immunoglobulin is an immunoglobulin G (IgG), an immunoglobulin A (IgA), or an immunoglobulin M (IgM). In some embodiments, the IgG is a subclass 1 IgG (IgG1) or a subclass 2 IgG (IgG2).

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1D. Discovery of PVP-179 in a human PBMC high-throughput screen. FIG. 1A: A graphical overview depicting screen approach. From a high-throughput screen previously conducted, 310 molecules were identified to be activators of either THP-1 cells (monocyte cell line) or primary human adult peripheral blood mononuclear cells (PBMCs). These 310 molecules were assembled into a stimulation plate and then applied to the PBMCs of 4 elderly donors (cultured with 10% autologous plasma). FIG. 1B: The bar graph displays the 45 highest calculated Z scores from the 310 molecules screened. Z scores were averaged across all four elderly donors. The Z score and chemical structure for PVP-179 are indicated. FIG. 1C: Human adult or elderly PBMCs (cultured with 10% autologous plasma) were stimulated with PVP-179 in a two-point concentration titration manner. After 18 hours of incubation, supernatant was processed for ELISA-based measurement of TNF production. N=4 donors. FIG. 1D: As in FIG. 1C, the supernatants were collected, and 14 cytokines were measured by multiplex assay. The radar plot shows the average pg/mL (in log scale) of cytokine production from 25 μM of PVP-179 stimulation in elderly or adult PBMCs compared with 25 μM of R848 stimulation in adult PBMCs. N=4 donors each.

FIGS. 2A-2B. PVP-179 demonstrates adjuvanticity with spike protein in elder mice. FIG. 2A: Adult (˜12 weeks) BALB/c mice were immunized with saline, spike protein (rSpike; 1 g), Spike protein in combination with aluminum hydroxide (alumOH; 100 g), ASO1, or PVP-179 (100 nmoles). Mice were immunized at Day 0 and subsequently administered a booster immunization at Day 14. Serum was collected at Day 28 to measure antibody titers for spike-specific IgG and subtypes. Overall IgG, IgG1, and IgG2 levels are presented as box and whisker plots representing 4 mice/group at Day 28. *, **, *** respectively indicate p≤0.05, 0.01 and 0.001 for comparisons between different experimental groups. FIG. 2B: Aged (˜52 weeks) BALB/c mice were immunized as in FIG. 2A. Serum was collected at Day 28 to measure antibody titers for spike-specific IgG and subtypes. Overall IgG, IgG1, and IgG2 levels are presented as box and whisker plots representing 4 mice/group at Day 28. *, **, *** respectively indicate p≤0.05, 0.01 and 0.001.

FIGS. 3A-3E. PVP-179 is a TLR8 agonist. FIG. 3A: PVP-179 was sent to InvivoGen for PRR ligand screening service. Various cells lines were utilized to screen for PVP-179-induced (33 M) activation of TLRs, NOD1/2, RIG-I/MDA-5, Dectin-1 or Mincle receptor. The heat-map depicts percentage activity compared to the positive control for each receptor. FIG. 3B: PVP-179 induced activity (% activity compared to the positive control: CL075 at 1 μg/mL) in HEK-293 cells specifically expressing hTLR8. In brief, these cells have an NF-κB/AP-1-inducible secreted embryonic alkaline phosphatase (SEAP) reporter gene which allows monitoring of signaling via TLR8. After incubation for 16-24 hours, supernatant harvested from the cells is mixed with media containing SEAP-substrate and the optical density (OD) is measured at 650 nm using a spectrophotometer. FIG. 3C: As in FIG. 3B, HEK cells specifically expressing hTLR2, hTLR7, or hTLR8, in addition to the HEK-Null cell line carrying only the reporter gene, were stimulated with the indicated concentrations of PVP-179. Supernatants were harvested after 20-24 hours to assess TLR dependent NF-κB activation. PVP-179 activity is expressed as a percentage of NF-κB induced in the cells stimulated with TNF (positive control for each cell line). FIG. 3D: Cells were incubated with the TLR8 specific inhibitor (CUCPT-9a, 1 μM) for 30-60 minutes. After inhibition, cells were stimulated with TNF, TL8-506 (TLR8 specific agonist) and different concentrations of PVP-179. The supernatants were harvested after 20-24 hours and SEAP production (to assess NF-κB activity) was measured using QUANTIBlue assay. The graphs represent values corresponding to percentage of TNF activity. N=3 independent experiments.

FIG. 3E: As in FIG. 3D, PBMCs isolated from human adult donors were cultured with 10% autologous plasma and incubated with CUCPT-9a. After inhibition, cells were stimulated with DMSO, TL8-506 (TLR8 specific agonist), LPS (TLR4 agonist; negative control) and different concentrations of PVP-179. Supernatants were harvested after 20-24 hours and analyzed for production of TNF via ELISA. N=6 donors.

FIGS. 4A-4D. PVP-179 activates human macrophages and dendritic cells. FIGS. 4A-4B: Murine mononuclear cells were isolated from bone marrow and induced differentiation into dendritic cells (with GM-CSF and IL-4). After 5 days of culturing, these bone-marrow-derived dendritic cells (BMDCs) were stimulated with PVP-179 or MPLA for 18 hours at indicated concentrations. The supernatants were analyzed via ELISA where each dot in the graph represents the mean production of mTNF (FIG. 4A) and mIL-6 (FIG. 4B). N=6 for PVP-179, N=3 for MPLA. Two-way Anova was performed to determine statistical significance. The comparisons shown are compared to control group (0 M PVP-179). FIGS. 4C-4D: PBMCs were isolated from blood of adult human donors. CD14+ monocytes were specifically isolated from the PBMCs and were then cultured in 10% autologous plasma and induced to differentiate into macrophages with M-CSF (FIG. 4C), or dendritic cells with GM-CSF and IL-4 (FIG. 4D). After 5 days, cells were harvested from each differentiated cell type (macrophages: Mφ; dendritic cells: MoDCs) and a 96-well plate was setup with 100,000 cells/well. Cells were then stimulated with indicated concentrations of PVP-179 or R848 (33 μM) or LPS (100 ng/mL). After 20-24 hours of incubation, supernatants were harvested and applied to TNF ELISA. N=2 donors.

### DETAILED DESCRIPTION OF CERTAIN EMBODIMENTS

Some aspects of the present disclosure are based, at least in part, on the finding that the compound disclosed herein induces a T helper type 1 (Th1) immune response associated with robust titration-dependent cytokine production in human peripheral blood mononuclear cells (PBMCs) in vitro and act as adjuvants in vivo. As described herein, the compound may be used for modifying human immune responses, including innate and adaptive immune responses. In some embodiments, the compound is used as an adjuvant in vaccines. Adjuvants can enhance, prolong, and modulate immune responses to vaccinal antigens to maximize protective immunity.

In some aspects, using the compound as a vaccine adjuvant enables effective immunization in vulnerable populations (e.g., neonates, elderly, or immunocompromised individuals). In some embodiments, the compound is used in the treatment (both prophylactically or therapeutically) of infectious diseases, cancers, or allergies.

### Immunostimulatory Compound

In some embodiments, the compound described herein is used in methods of enhancing an immune response in a subject in need thereof, comprising administering to the subject an effective amount of the compound. In some embodiments, the compound described herein is used in methods of treating a disease or reducing the risk of a disease (e.g., proliferative disease, inflammatory disease, autoimmune disease, infectious disease, or chronic disease) in a subject in need thereof, comprising administering to the subject an effective amount of the compound. In some embodiments, the compound is an adjuvant. In one aspect, the compound is the compound of Formula (I):

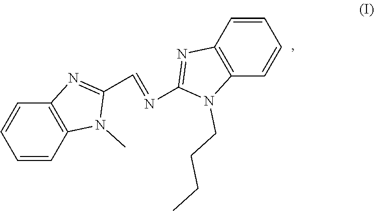 [SMILES: CCCCn1c(/N=C/c2nc3ccccc3n2C)nc2ccccc21]

and pharmaceutically acceptable salts, solvates, hydrates, polymorphs, co-crystals, tautomers, stereoisomers, isotopically labeled derivatives, and prodrugs thereof. In some embodiments, the compound of Formula (I) and any pharmaceutically acceptable salt, solvate, hydrate, polymorph, co-crystal, tautomer, stereoisomer, isotopically labeled derivative, and prodrug thereof may be interchangeably referred to as a compound, a molecule, a small molecule, a pharmacophore, or any other equivalent term that is known in the art.

Provided herein are uses for the compound of Formula (I) as an adjuvant in a vaccine and/or for use in enhancing an immune response in a subject in need thereof. In some embodiments, the compound of Formula (I) described herein is for use as an adjuvant in a vaccine. In some embodiments, the compound of Formula (I) described herein is for use in enhancing an immune response in a subject in need thereof.

### Antigens

Some aspects of the present invention relate to compositions comprising an antigen and the compound of Formula (I). An “antigen” refers to an entity that is bound by an antibody or receptor, or an entity that induces the production of an antibody. In some embodiments, an antigen increases the production of antibodies that specifically binds to the antigen at one or more epitopes. In some embodiments, an antigen comprises a protein or polypeptide. Such a protein or peptide is referred to herein as an “immunogenic polypeptide.” In some embodiments, the term “antigen” encompasses nucleic acids (e.g., DNA or RNA molecules) that are immunogenic or encode immunogenic polypeptides. In some embodiments, the antigen is from a microbial pathogen. For example, the antigen may comprise parts (coats, capsules, cell walls, flagella, fimbriae, and toxins) of bacteria, viruses, fungi, and other microorganisms. In some embodiments, the antigen is a cancer-specific antigen.

In some embodiments, a protein or polypeptide antigen is a wild type protein or polypeptide. In some embodiments, a protein or polypeptide antigen is a polypeptide variant of a wild type protein or polypeptide. The term “polypeptide variant” refers to molecules which differ in their amino acid sequence from a wild type (native) or reference sequence. Amino acid sequence variants may possess substitutions, deletions, and/or insertions at certain positions within the amino acid sequence, as compared to a wild type (native) or reference sequence. One measure of the degree of similarity between a polypeptide variant and a wild type (native) or reference sequence is the overall percentage of amino acids in the polypeptide variant that are identical to (i.e., occurring in the same position and same amino acid type as) the corresponding amino acids of the wild type (native) or reference sequence (i.e., “% identity”). In some embodiments, polypeptide variants possess at least 50% identity to a native or reference sequence. In some embodiments, variants share at least 70%, at least 80%, at least 90%, at least 95%, or at least 99% identity with a native or reference sequence.

In some embodiments, a polypeptide variant encompasses covalent variants and derivatives. The term “derivative” is used synonymously with the term “variant” but generally refers to a molecule that has been modified and/or changed in any way relative to a reference molecule or starting molecule.

In some embodiments, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the polypeptide, as for example, expression of the polypeptide as part of a larger sequence which is soluble or linked to a solid support.

In some embodiments, a polypeptide variant comprises at least one substituted amino acid residue, in which an amino acid within a wild type (native) or initial polypeptide sequence is removed and a different amino acid is inserted in its place at the same position. Substitutions may be single, where only one amino acid in the molecule has been substituted, or they may be multiple, where two or more amino acids have been substituted in the same molecule. In some embodiments, the polypeptide that includes 2, 3, 4, 5, 6, 7, 8, 9, 10, or more substitutions compared to a reference protein. In some embodiments, a polypeptide comprising substituted amino acids is an antigen.

In some embodiments, a substitution is a conservative amino acid substitution. The term “conservative amino acid substitution” refers to the substitution of an amino acid that is normally present in a sequence with a different amino acid of similar size, charge, and/or polarity.

Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In some embodiments, protein fragments, functional protein domains, and homologous proteins are used as antigens in accordance with the present disclosure. For example, an antigen may comprise any protein fragment (meaning a polypeptide sequence which is at least one amino acid residue shorter than a reference polypeptide sequence but otherwise identical) of a reference protein that is, for example, 10, 20, 30, 40, 50, 60, 70, 80, 90, 100 or greater than 100 amino acids in length. In another example, a polypeptide variant that includes a stretch of 20, 30, 40, 50, or 100 amino acids which are 40%, 50%, 60%, 70%, 80%, 90%, 95%, or 100% identical to a reference protein (e.g., a protein from a microbial pathogen) herein can be utilized in accordance with the disclosure.

In some embodiments, an antigen comprises more than one (e.g., 2, 3, 4, 5, 6, 7, 8, 9, 10, or more) immunogenic proteins or polypeptides. In some embodiments, the more than one immunogenic proteins or polypeptides are derived from one protein (e.g., different fragments or polypeptide variants derived from one protein). In some embodiments, the more than one (e.g., from 2, 3, 4, 5, 6, 7, 8, 9, 10, or more proteins) immunogenic proteins or polypeptides are derived from multiple proteins.

In some embodiments, an antigen comprises a nucleic acid encoding an immunogenic protein or polypeptide. In some embodiments, the antigen comprises an immunogenic protein or polypeptide and a nucleic acid encoding the immunogenic protein or polypeptide. The term “nucleic acid” or “polynucleotide,” in its broadest sense, includes any compound and/or substance that comprises a polymer of nucleotides. Nucleic acids encoding immunogenic proteins or polypeptides typically comprise an open reading frame (ORF), and one or more regulatory sequences. Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA), or chimeras or combinations thereof.

In some embodiments, a nucleic acid encoding an immunogenic polypeptide is a DNA (e.g., a DNA expression vector for an immunogenic protein or polypeptide). In some embodiments, the nucleic acid encoding the immunogenic polypeptide is a RNA (e.g., a messenger RNA). A “messenger RNA” (mRNA) refers to any polynucleotide that encodes a (at least one) polypeptide (e.g., a naturally occurring, non-naturally occurring, or modified polymer of amino acids) and can be translated to produce the encoded polypeptide in vitro, in vivo, in situ, or ex vivo. The basic components of an mRNA molecule typically include at least one coding region, a 5′ untranslated region (UTR), a 3′ UTR, a 5′ cap, and a poly-A tail.

In some embodiments, the coding region of a nucleic acid (e.g., DNA or RNA) encoding an immunogenic polypeptide is codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art. Non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally occurring or wild-type sequence (e.g., a naturally occurring or wild-type mRNA sequence encoding an immunogenic protein or polypeptide). In some embodiments, a codon optimized sequence shares less than 90% sequence identity to a naturally occurring or wild-type sequence (e.g., a naturally occurring or wild-type mRNA sequence encoding an immunogenic protein or polypeptide). In some embodiments, a codon optimized sequence shares less than 85% sequence identity to a naturally occurring or wild-type sequence (e.g., a naturally occurring or wild-type mRNA sequence encoding an immunogenic protein or polypeptide). In some embodiments, a codon optimized sequence shares less than 80% sequence identity to a naturally occurring or wild-type sequence (e.g., a naturally occurring or wild-type mRNA sequence encoding an immunogenic protein or polypeptide). In some embodiments, a codon optimized sequence shares less than 75% sequence identity to a naturally occurring or wild-type sequence (e.g., a naturally occurring or wild-type mRNA sequence encoding an immunogenic protein or polypeptide).

In some embodiments, a nucleic acid encoding an immunogenic protein or polypeptide comprises one or more chemical modifications. The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population.

In some embodiments, a nucleic acid (e.g., DNA or RNA) encoding an immunogenic polypeptide comprises various (more than one) different modifications. In some embodiments, a particular region of a nucleic acid (e.g., DNA or RNA) contains one, two, or more (optionally different) nucleoside or nucleotide modifications. In some embodiments, a modified nucleic acid (e.g., DNA or RNA), when introduced into a cell or organism, exhibits reduced degradation in the cell or organism, respectively, relative to an unmodified nucleic acid. In some embodiments, a modified nucleic acid (e.g., DNA or RNA), when introduced into a cell or organism, may exhibit reduced immunogenicity in the cell or organism, respectively (e.g., a reduced innate response).

A modified nucleic acid (e.g., DNA or RNA) may comprise modifications that are naturally occurring or non-naturally occurring, or the polynucleotide may comprise a combination of naturally occurring and non-naturally occurring modifications. A nucleic acid encoding an immunogenic polypeptide described herein may include any useful modification, for example, of a sugar, a nucleobase, or an inter-nucleoside linkage (e.g., to a linking phosphate, to a phosphodiester linkage or to the phosphodiester backbone). Modified nucleic acid (e.g., DNA or RNA), in some embodiments, comprise non-natural modified nucleotides that are introduced during synthesis or post-synthesis of the polynucleotides to achieve desired functions or properties. The modifications may be present on inter-nucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

In some embodiments, a chemically modified nucleic acid comprises one or more modified nucleosides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

In some embodiments, a modified nucleobase is a modified cytosine. Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and nucleosides having a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

In some embodiments, an antigen of the present disclosure is from a microbial pathogen, e.g., from a bacterium, a mycobacterium, a fungus, a virus, a parasite, or a prion. For example, the antigen may comprise a protein or polypeptide, a nucleic acid, or a nucleic acid encoding a protein or polypeptide from the microbial pathogen. In some embodiments, the antigen may comprise a microbial pathogen (e.g., a bacterial cell, a viral particle, or a fungus cell). In some embodiments, the microbial pathogen cell is live or killed. In some embodiments, the microbial pathogen is attenuated its pathogenicity. An attenuated microbial pathogen may elicit immune response but does not cause the disease that a wild-type microbial pathogen would cause.

Exemplary, non-limiting bacterial taxa, species, and strains, suitable for use in some embodiments of this disclosure include: Escherichia spp., Enterobacter spp. (e.g., Enterobacter cloacae), Salmonella spp. (e.g., Salmonella enteritidis, Salmonella typhi), Shigella spp., Pseudomonas spp. (e.g., Pseudomonas aeruginosa, Pseudomonas pachastrellae, Pseudomonas stutzeri), Moraxella spp. (e.g., Moraxella catarrhalis), Neisseria spp. (e.g., Neisseria gonorrhoeae, Neisseria meningitidis), Helicobacter spp. (e.g., Helicobacter pylori) Stenotrophomonas spp., Vibrio spp. (e.g., Vibrio cholerae), Legionella spp. (Legionella pneumophila), Hemophilus spp. (e.g., Hemophilus influenzae), Klebsiella spp. (e.g., Klebsiella pneumoniae), Proteus spp. (e.g., Proteus mirabilis), Serratia spp. (Serratia marcescens), Streptococcus spp., Staphylococcus spp., Corynebacterium spp., Listeria spp., and Clostridium spp., Bacillus spp. (e.g., Bacillus anthracis) Bordetella spp. (e.g., Bordetella pertussis); Borrelia spp. (e.g., Borrelia burgdorferi); Brucella spp. (e.g., Brucella abortus, Brucella canis, Brucella melitensis, Brucella suis); Campylobacter spp. (e.g., Campylobacter jejuni); Chlamydia spp. and Chlamydophila spp. (e.g., Chlamydia pneumoniae, Chlamydia trachomatis, Chlamydophila psittaci); Clostridium spp. (e.g., Clostridium botulinum, Clostridium difficile, Clostridium perfringens, Clostridium tetani); Corynebacterium spp. (e.g., Corynebacterium diphtheriae); Enterococcus spp. (e.g., Enterococcus faecalis, Enterococcus faecium); Escherichia spp. (e.g., Escherichia coli, Enterotoxic E. coli, enteropathogenic E. coli; E. coli O157:H7); Francisella spp. (e.g., Francisella tularensis); Haemophilus spp. (e.g., Haemophilus influenzae); Helicobacter spp. (e.g., Helicobacter pylori); Legionella spp. (e.g., Legionella pneumophila); Leptospira spp. (e.g., Leptospira interrogans); Listeria spp. (e.g., Listeria monocytogenes); Mycobacterium spp. (e.g., Mycobacterium leprae, Mycobacterium tuberculosis, Mycobacterium ulcerans); Mycoplasma spp. (e.g., Mycoplasma pneumoniae); Neisseria spp. (e.g., Neisseria gonorrhoeae, Neisseria meningitidis); Pseudomonas spp. (e.g., Pseudomonas aeruginosa); Rickettsia spp. (e.g., Rickettsia rickettsii); Salmonella spp. (e.g., Salmonella typhi, Salmonella typhimurium); Shigella spp. (e.g., Shigella sonnei); Staphylococcus spp. (e.g., Staphylococcus aureus, Staphylococcus epidermidis, Staphylococcus saprophyticus); Streptococcus spp. (e.g., Streptococcus agalactiae, Streptococcus pneumoniae, Streptococcus pyogenes); Treponema spp. (e.g., Treponema pallidum); Pseudodiomarina spp. (e.g., P. maritima); Marinobacter spp. (e.g., Marinobacter hydrocarbonoclasticus, Marinobacter vinifirmus) Alcanivorax spp. (e.g., alcanivorax dieselolei); Acetinobacter spp. (e.g., A. venetianus); Halomonas spp. (e.g., H. shengliensis); Labrenzia spp.; Microbulifer spp. (e.g., M. schleiferi); Shewanella spp. (e.g., S. algae); Vibrio spp. (e.g., Vibrio cholerae, Vibrio alginolyticus, Vibrio hepatarius); and Yersinia spp. (e.g., Yersinia pestis).

In some embodiments, the bacterium is Bacillus anthracis (causing anthrax), Bordetella pertussis (causing whooping cough), Corynebacterium diphtheriae (causing diphtheria), Clostridium tetani (causing tetanus), Haemophilus influenzae type b, pneumococcus (causing pneumococcal infections), Streptococcus spp., Staphylococci spp. (including Group A or B streptococci), Mycobacterium spp. (e.g., Mycobacterium tuberculosis), Neiserria spp. (e.g., Neiserria meningitidis-causing meningococcal disease), Salmonella typhi (causing typhoid), Vibrio cholerae (causing Cholera), or Yersinia pestis (causing plague).

In some embodiments, an antigen is derived from a Gram-negative bacterium. In some embodiments, the antigen comprises a lipopolysaccharide endotoxin (LPS) from a Gram-negative bacterium. A “lipopolysaccharide endotoxin (LPS)” refers to a large molecule consisting of a lipid and a polysaccharide composed of O-antigen, outer core and inner core joined by a covalent bond. LPS is found in the outer membrane of Gram-negative bacteria. Non-limiting examples of gram-negative bacterial species include: Neisseria species including Neisseria gonorrhoeae and Neisseria meningitidis, Branhiamella species including Branhamelia catarrhalis, Escherichia species including Escherichia coli, Enterobacter species, Proteus species including Proteus mirabilis, Pseudomonas species including Pseudomonas aeruginosa, Pseudomonas mallei, and Pseudomonas pseudomallei, Klebsiella species including Klebsiella pneumoniae, Salmonella species, Shigella species, Serratia species, Acinetobacter species; Haemophilus species including Haemophilus influenzae and Haemophilus ducreyi, Brucella species. Yersinia species including Yersinia pestis and Yersinia enterocolitica, Francisella species including Francisella tularensis, Pasteurella species including Pasteurella muliocida, Vibrio cholerae, Flavobacterium species, meningosepticum, Campylobacter species including Campylobacter jejuni, Chlamydia species including Chlamydia trachomatis, Bacteroides species (oral, pharyngeal) including Bacteroides fragilis, Fusobacterium species including Fusobacterium nucleatum, Calymmatobacterium granulomatis, Streptobacillus species including Streprobacillus moniliformiis, Legionella species including Legionella pneumophila.

In some embodiments, an antigen is derived from a Gram-positive bacterium. Exemplary Gram-positive bacteria include, but are not limited to, Staphylococcus spp., Streptococcus spp., Micrococcus spp., Peptococcus spp., Peptostreptococcus spp., Enterococcus spp., Bacillus spp., Clostridium spp., Lactobacillus spp., Listeria spp., Erysipelothrix spp., Propionibacterium spp., Eubacterium spp., Corynebacterium spp, Capnocybophaga spp., Bifidobacterium spp., and Gardnerella spp., In some embodiments, the Gram-positive bacterium is a bacterium of the phylum Firmicutes. In some embodiments, the Gram-positive bacterium is Streptococcus.

Other types of bacteria include acid-fast bacilli, spirochetes, and actinomycetes. Examples of acid-fast bacilli include Mycobacterium species including Mycobacterium tuberculosis and Mycobocteriumn leprae. Examples of spirochetes include Treponema species including Treponema pallidum, Treponema pertenue, Borrelia species including Borrelia burgdorferi (Lyme disease), and Borrelia recurrentis, and Leptospira species. Examples of actinomycetes include: Actinomyces species including Actinomyces israelii, and Nocardia species including Nocardia asteroides.

Examples of viruses include but are not limited to: Retroviruses, human immunodeficiency viruses including HIV-1, HDTV-III, LAVE, HTLV-III/LAV, HIV-JJJ, HIV-LP, Cytomegaloviruses (CMV), Picornaviruses, polio viruses, hepatitis A virus, enteroviruses, human Coxsackie viruses, rhinoviruses, echoviruses, Calciviruses, Togaviruses, equine encephalitis viruses, rubella viruses, Flaviruses, dengue viruses, encephalitis viruses, yellow fever viruses, Coronaviruses, Rhabdoviruses, vesicular stomatitis viruses, rabies viruses, Filoviruses, Ebola virus, Paramyxoviruses, parainfluenza viruses, mumps virus, measles virus, respiratory syncytial virus (RSV), Orthomyxoviruses, influenza viruses, Bungaviruses, Hantaan viruses, phleboviruses and Nairo viruses, Arena viruses, hemorrhagic fever viruses, reoviruses, orbiviruses, rotaviruses, Birnaviruses, Hepadnaviruses, Hepatitis B virus, parvoviruses, Papovaviridae, papilloma viruses, polyoma viruses, Adenoviruses, Herpesviruses including herpes simplex virus 1 and 2, varicella zoster virus, Poxviruses, variola viruses, vaccinia viruses, Irido viruses, African swine fever virus, delta hepatitis virus, non-A, non-B hepatitis virus, Hepatitis C, Norwalk viruses, astroviruses, and unclassified viruses. In some embodiments, the virus is adenovirus, enterovirus such as poliomyelitis (polio), Ebola virus, herpes viruses such as herpes simplex virus, cytomegalovirus and varicella-zoster (chickenpox and shingles), measles, mumps, rubella, hepatitis-A, -B, or -C, human papilloma virus, Influenza virus, rabies, Japanese encephalitis, rotavirus, human immunodeficiency virus (HIV), respiratory syncytial virus (RSV), smallpox, yellow fever, or Zika Virus.

In some embodiments, an antigen comprises a viral protein and/or a nucleic acid encoding a viral protein (e.g., a viral structural or non-structural protein). In some embodiments, the antigen comprises a nucleic acid encoding the viral genome. In some embodiments, the viral genome is modified to produce a modified virus that is attenuated. In some embodiments, the antigen comprises a live attenuated, inactivated, or killed virus.

In some embodiments, an antigen comprises a protein and/or a nucleic acid encoding a viral protein from a Beta Coronavirus, such as MERS-CoV, SARS-CoV-1, or SARS-CoV-2. In some embodiments, the antigen comprises a spike protein, envelope protein, membrane protein, or nucleocapsid protein from a Beta Coronavirus, and/or a nucleic acid encoding a spike protein, envelope protein, membrane protein, or nucleocapsid protein from a Beta Coronavirus. In some embodiments, the antigen comprises an immunogenic fragment of a protein from a Beta Coronavirus, and/or a nucleic acid encoding an immunogenic fragment of a protein from a Beta Coronavirus (e.g., a receptor binding domain (RBD) of Beta coronavirus spike protein). In some embodiments, an antigen comprises a live attenuated, inactivated, or killed MERS-CoV, SARS-CoV-1, or SARS-CoV-2 virus.

Amino acid sequences of example Beta coronavirus protein antigens described herein are provided in Table 1:

TABLE 1
Beta Coronavirus protein antigens

| Antigen | Amino Acid Sequence |
|---|---|
| SARS-CoV-1 | MFIFLLFLTLTSGSDLDRCTTFDDVQAPNY |
| Spike Protein | TQHTSSMRGVYYPDEIFRSDTLYLTQDLFL |
| (SEQ ID NO: 1) | PFYSNVTGFHTINHTFGNPVIPFKDGIYFA |
| | ATEKSNVVRGWVFGSTMNNKSQSVIIINNS |
| | TNVVIRACNFELCDNPFFAVSKPMGTQTHT |
| | MIFDNAFNCTFEYISDAFSLDVSEKSGNFK |
| | HLREFVFKNKDGFLYVYKGYQPIDVVRDLP |
| | SGFNTLKPIFKLPLGINITNFRAILTAFSP |
| | AQDIWGTSAAAYFVGYLKPTTFMLKYDENG |
| | TITDAVDCSQNPLAELKCSVKSFEIDKGIY |
| | QTSNFRVVPSGDVVRFPNITNLCPFGEVFN |
| | ATKFPSVYAWERKKISNCVADYSVLYNSTF |
| | FSTFKCYGVSATKLNDLCFSNVYADSFVVK |
| | GDDVRQIAPGQTGVIADYNYKLPDDFMGCV |
| | LAWNTRNIDATSTGNYNYKYRYLRHGKLRP |
| | FERDISNVPFSPDGKPCTPPALNCYWPLND |
| | YGFYTTTGIGYQPYRVVVLSFELLNAPATV |
| | CGPKLSTDLIKNQCVNFNFNGLTGTGVLTP |
| | SSKRFQPFQQFGRDVSDFTDSVRDPKTSEI |
| | LDISPCSFGGVSVITPGTNASSEVAVLYQD |
| | VNCTDVSTAIHADQLTPAWRIYSTGNNVFQ |
| | TQAGCLIGAEHVDTSYECDIPIGAGICASY |
| | HTVSLLRSTSQKSIVAYTMSLGADSSIAYS |
| | NNTIAIPTNFSISITTEVMPVSMAKTSVDC |
| | NMYICGDSTECANLLLQYGSFCTQLNRALS |
| | GIAAEQDRNTREVFAQVKQMYKTPTLKYFG |
| | GFNFSQILPDPLKPTKRSFIEDLLFNKVTL |
| | ADAGFMKQYGECLGDINARDLICAQKFNGL |
| | TVLPPLLTDDMIAAYTAALVSGTATAGWTF |
| | GAGAALQIPFAMQMAYRFNGIGVTQNVLYE |
| | NQKQIANQFNKAISQIQESLTTTSTALGKL |
| | QDVVNQNAQALNTLVKQLSSNFGAISSVLN |
| | DILSRLDKVEAEVQIDRLITGRLQSLQTYV |
| | TQQLIRAAEIRASANLAATKMSECVLGQSK |
| | RVDFCGKGYHLMSFPQAAPHGVVFLHVTYV |
| | PSQERNFTTAPAICHEGKAYFPREGVFVFN |
| | GTSWFITQRNFFSPQIITTDNTFVSGNCDV |
| | VIGIINNTVYDPLQPELDSFKEELDKYFKN |
| | HTSPDVDLGDISGINASVVNIQKEIDRLNE |
| | VAKNLNESLIDLQELGKYEQYIKWPWYVWL |
| | GFIAGLIAIVMVTILLCCMTSCCSCLKGAC |
| | SCGSCCKFDEDDSEPVLKGVKLHYT |
| SARS-CoV-1 | RVVPSGDVVRFPNITNLCPFGEVFNATKFP |
| Spike Protein | SVYAWERKKISNCVADYSVLYNSTFFSTFK |
| receptor | CYGVSATKLNDLCFSNVYADSFVVKGDDVR |
| binding | QIAPGQTGVIADYNYKLPDDFMGCVLAWNT |
| domain (SEQ | RNIDATSTGNYNYKYRYLRHGKLRPFERDI |
| ID NO: 2) | SNVPFSPDGKPCTPPALNCYWPLNDYGFYT |
| | TTGIGYQPYRVVVLSFELLNAPATVCGPKL |
| | STDLIKNQCVNF |
| SARS-CoV-1 | MYSFVSEETGTLIVNSVLLFLAFVVFLLVT |
| Envelope | LAILTALRLCAYCCNIVNVSLVKPTVYVYS |
| Protein (SEQ | RVKNLNSSEGVPDLLV |
| ID NO: 3) | |
| SARS-CoV-1 | MADNGTITVEELKQLLEQWNLVIGFLFLAW |
| Membrane | IMLLQFAYSNRNRFLYIIKLVFLWLLWPVT |
| Protein (SEQ | LACFVLAAVYRINWVTGGIAIAMACIVGLM |
| ID NO: 4) | WLSYFVASFRLFARTRSMWSFNPETNILLN |
| | VPLRGTIVTRPLMESELVIGAVIIRGHLRM |
| | AGHSLGRCDIKDLPKEITVATSRTLSYYKL |
| | GASQRVGTDSGFAAYNRYRIGNYKLNTDHA |
| | GSNDNIALLVQ |
| SARS-CoV-1 | MSDNGPQSNQRSAPRITFGGPTDSTDNNQN |
| Nucleocapsid | GGRNGARPKQRRPQGLPNNTASWFTALTQH |
| Protein (SEQ | GKEELRFPRGQGVPINTNSGPDDQIGYYRR |
| ID NO: 5) | ATRRVRGGDGKMKELSPRWYFYYLGTGPEA |
| | SLPYGANKEGIVWVATEGALNTPKDHIGTR |
| | NPNNNAATVLQLPQGTTLPKGFYAEGSRGG |
| | SQASSRSSSRSRGNSRNSTPGSSRGNSPAR |
| | MASGGGETALALLLLDRLNQLESKVSGKGQ |
| | QQQGQTVTKKSAAEASKKPRQKRTATKQYN |
| | VTQAFGRRGPEQTQGNFGDQDLIRQGTDYK |
| | HWPQIAQFAPSASAFFGMSRIGMEVTPSGT |
| | WLTYHGAIKLDDKDPQFKDNVILLNKHIDA |
| | YKTFPPTEPKKDKKKKTDEAQPLPQRQKKQ |
| | PTVTLLPAADMDDFSRQLQNSMSGASADST |
| | QA |
| SARS-CoV-2 | MFVFLVLLPLVSSQCVNLTTRTQLPPAYTN |
| Spike Protein | SFTRGVYYPDKVFRSSVLHSTQDLFLPFFS |
| (SEQ ID NO: 6) | NVTWFHAIHVSGTNGTKRFDNPVLPFNDGV |
| | YFASTEKSNIIRGWIFGTTLDSKTQSLLIV |
| | NNATNVVIKVCEFQFCNDPFLGVYYHKNNK |
| | SWMESEFRVYSSANNCTFEYVSQPFLMDLE |
| | GKQGNFKNLREFVFKNIDGYFKIYSKHTPI |
| | NLVRDLPQGFSALEPLVDLPIGINITRFQT |
| | LLALHRSYLTPGDSSSGWTAGAAAYYVGYL |
| | QPRTFLLKYNENGTITDAVDCALDPLSETK |
| | CTLKSFTVEKGIYQTSNFRVQPTESIVRFP |
| | NITNLCPFGEVFNATRFASVYAWNRKRISN |
| | CVADYSVLYNSASFSTFKCYGVSPTKLNDL |
| | CFTNVYADSFVIRGDEVRQIAPGQTGKIAD |
| | YNYKLPDDFTGCVIAWNSNNLDSKVGGNYN |
| | YLYRLFRKSNLKPFERDISTEIYQAGSTPC |
| | NGVEGFNCYFPLQSYGFQPTNGVGYQPYRV |
| | VVLSFELLHAPATVCGPKKSTNLVKNKCVN |
| | FNFNGLTGTGVLTESNKKFLPFQQFGRDIA |
| | DTTDAVRDPQTLEILDITPCSFGGVSVITP |
| | GTNTSNQVAVLYQDVNCTEVPVAIHADQLT |
| | PTWRVYSTGSNVFQTRAGCLIGAEHVNNSY |
| | ECDIPIGAGICASYQTQTNSPRRARSVASQ |
| | SIIAYTMSLGAENSVAYSNNSIAIPTNFTI |
| | SVTTEILPVSMTKTSVDCTMYICGDSTECS |
| | NLLLQYGSFCTQLNRALTGIAVEQDKNTQE |
| | VFAQVKQIYKTPPIKDFGGFNFSQILPDPS |
| | KPSKRSFIEDLLFNKVTLADAGFIKQYGDC |
| | LGDIAARDLICAQKFNGLTVLPPLLTDEMI |
| | AQYTSALLAGTITSGWTFGAGAALQIPFAM |
| | QMAYRFNGIGVTQNVLYENQKLIANQFNSA |
| | IGKIQDSLSSTASALGKLQDVVNQNAQALN |
| | TLVKQLSSNFGAISSVLNDILSRLDKVEAE |
| | VQIDRLITGRLQSLQTYVTQQLIRAAEIRA |
| | SANLAATKMSECVLGQSKRVDFCGKGYHLM |
| | SFPQSAPHGVVFLHVTYVPAQEKNFTTAPA |
| | ICHDGKAHFPREGVFVSNGTHWFVTQRNFY |
| | EPQIITTDNTFVSGNCDVVIGIVNNTVYDP |
| | LQPELDSFKEELDKYFKNHTSPDVDLGDIS |
| | GINASVVNIQKEIDRLNEVAKNLNESLIDL |
| | QELGKYEQYIKWPWYIWLGFIAGLIAIVMV |
| | TIMLCCMTSCCSCLKGCCSCGSCCKFDEDD |
| | SEPVLKGVKLHYT |
| SARS-CoV-2 | RVQPTESIVRFPNITNLCPFGEVFNATRFA |
| Spike Protein | SVYAWNRKRISNCVADYSVLYNSASFSTFK |
| receptor | CYGVSPTKLNDLCFTNVYADSFVIRGDEVR |
| binding | QIAPGQTGKIADYNYKLPDDFTGCVIAWNS |
| domain (SEQ | NNLDSKVGGNYNYLYRLFRKSNLKPFERDI |
| ID NO: 7) | STEIYQAGSTPCNGVEGENCYFPLQSYGFQ |
| | PTNGVGYQPYRVVVLSFELLHAPATVCGPK |
| | KSTNLVKNKCVNF |
| SARS-CoV-2 | MYSFVSEETGTLIVNSVLLFLAFVVFLLVT |
| Envelope | LAILTALRLCAYCCNIVNVSLVKPSFYVYS |
| Protein (SEQ | RVKNLNSSRVPDLLV |
| ID NO: 8) | |
| SARS-CoV-2 | MADSNGTITVEELKKLLEQWNLVIGFLFLT |
| Membrane | WICLLQFAYANRNRFLYIIKLIFLWLLWPV |
| Protein (SEQ | TLACFVLAAVYRINWITGGIAIAMACLVGL |
| ID NO: 9) | MWLSYFIASFRLFARTRSMWSFNPETNILL |
| | NVPLHGTILTRPLLESELVIGAVILRGHLR |
| | IAGHHLGRCDIKDLPKEITVATSRTLSYYK |
| | LGASQRVAGDSGFAAYSRYRIGNYKLNTDH |
| | SSSSDNIALLVQ |
| SARS-CoV-2 | MSDNGPQNQRNAPRITFGGPSDSTGSNQNG |
| Nucleocapsid | ERSGARSKQRRPQGLPNNTASWFTALTQHG |
| Protein (SEQ | KEDLKFPRGQGVPINTNSSPDDQIGYYRRA |
| ID NO: 10) | TRRIRGGDGKMKDLSPRWYFYYLGTGPEAG |
| | LPYGANKDGIIWVATEGALNTPKDHIGTRN |
| | PANNAAIVLQLPQGTTLPKGFYAEGSRGGS |
| | QASSRSSSRSRNSSRNSTPGSSRGTSPARM |
| | AGNGGDAALALLLLDRLNQLESKMSGKGQQ |
| | QQGQTVTKKSAAEASKKPRQKRTATKAYNV |
| | TQAFGRRGPEQTQGNFGDQELIRQGTDYKH |
| | WPQIAQFAPSASAFFGMSRIGMEVTPSGTW |
| | LTYTGAIKLDDKDPNFKDQVILLNKHIDAY |
| | KTFPPTEPKKDKKKKADETQALPQRQKKQQ |
| | TVTLLPAADLDDFSKQLQQSMSSADSTQA |
| MERS-CoV | MIHSVFLLMFLLTPTESYVDVGPDSVKSAC |
| Spike protein | IEVDIQQTFFDKTWPRPIDVSKADGIIYPQ |
| (SEQ ID NO: | GRTYSNITITYQGLFPYQGDHGDMYVYSAG |
| 11) | HATGTTPQKLFVANYSQDVKQFANGFVVRI |
| | GAAANSTGTVIISPSTSATIRKIYPAFMLG |
| | SSVGNFSDGKMGRFFNHTLVLLPDGCGTLL |
| | RAFYCILEPRSGNHCPAGNSYTSFATYHTP |
| | ATDCSDGNYNRNASLNSFKEYFNLRNCTFM |
| | YTYNITEDEILEWFGITQTAQGVHLFSSRY |
| | VDLYGGNMFQFATLPVYDTIKYYSIIPHSI |
| | RSIQSDRKAWAAFYVYKLQPLTFLLDFSVD |
| | GYIRRAIDCGFNDLSQLHCSYESFDVESGV |
| | YSVSSFEAKPSGSVVEQAEGVECDFSPLLS |
| | GTPPQVYNFKRLVFTNCNYNLTKLLSLFSV |
| | NDFTCSQISPAAIASNCYSSLILDYFSYPL |
| | SMKSDLSVSSAGPISQFNYKQSFSNPTCLI |
| | LATVPHNLTTITKPLKYSYINKCSRLLSDD |
| | RTEVPQLVNANQYSPCVSTVPSTVWEDGDY |
| | YRKQLSPLEGGGWLVASGSTVAMTEQLQMG |
| | FGITVQYGTDTNSVCPKLEFANDTKIASQL |
| | GNCVEYSLYGVSGRGVFQNCTAVGVRQQRF |
| | VYDAYQNLVGYYSDDGNYYCLRACVSVPVS |
| | VIYDKETKTHATLFGSVACEHISSTMSQYS |
| | RSTRSMLKRRDSTYGPLQTPVGCVLGLVNS |
| | SLFVEDCKLPLGQSLCALPDTPSTLTPRSV |
| | RSVPGEMRLASIAFNHPIQVDQLNSSYFKL |
| | SIPTNFSFGVTQEYIQTTIQKVTVDCKQYV |
| | CNGFQKCEQLLREYGQFCSKINQALHGANL |
| | RQDDSVRNLFASVKSSQSSPIIPGFGGDFN |
| | LTLLEPVSISTGSRSARSAIEDLLFDKVTI |
| | ADPGYMQGYDDCMQQGPASARDLICAQYVA |
| | GYKVLPPLMDVNMEAAYTSSLLGSIAGVGW |
| | TAGLSSFAAIPFAQSIFYRLNGVGITQQVL |
| | SENQKLIANKFNQALGAMQTGFTTTNEAFR |
| | KVQDAVNNNAQALSKLASELSNTFGAISAS |
| | IGDIIQRLDVLEQDAQIDRLINGRLTTLNA |
| | FVAQQLVRSESAALSAQLAKDKVNECVKAQ |
| | SKRSGFCGQGTHIVSFVVNAPNGLYFMHVG |
| | YYPSNHIEVVSAYGLCDAANPTNCIAPVNG |
| | YFIKTNNTRIVDEWSYTGSSFYAPEPITSL |
| | NTKYVAPQVTYQNISTNLPPPLLGNSTGID |
| | FQDELDEFFKNVSTSIPNFGSLTQINTTLL |
| | DLTYEMLSLQQVVKALNESYIDLKELGNYT |
| | YYNKWPWYIWLGFIAGLVALALCVFFILCC |
| | TGCGTNCMGKLKCNRCCDRYEEYDLEPHKV |
| | HVH |
| MERS-CoV | ECDFSPLLSGTPPQVYNFKRLVFTNCNYNL |
| Spike protein | TKLLSLFSVNDFTCSQISPAAIASNCYSSL |
| receptor | ILDYFSYPLSMKSDLSVSSAGPISQFNYKQ |
| binding | SFSNPTCLILATVPHNLTTITKPLKYSYIN |
| domain (SEQ | KC |
| ID NO: 12) | |
| MERS-CoV | MLPFVQERIGLFIVNFFIFTVVCAITLLVC |
| Envelope | MAFLTATRLCVQCMTGFNTLLVQPALYLYN |
| protein (SEQ | TGRSVYVKFQDSKPPLPPDEWV |
| ID NO: 13) | |
| MERS-CoV | MSNMTQLTEAQIIAIIKDWNFAWSLIFLLI |
| Membrane | TIVLQYGYPSRSMTVYVFKMFVLWLLWPSS |
| protein (SEQ | MALSIFSAVYPIDLASQIISGIVAAVSAMM |
| ID NO: 14) | WISYFVQSIRLFMRTGSWWSFNPETNCLLN |
| | VPFGGTTVVRPLVEDSTSVTAVVTNGHLKM |
| | AGMHFGACDYDRLPNEVTVAKPNVLIALKM |
| | VKRQSYGTNSGVAIYHRYKAGNYRSPPITA |
| | DIELALLRA |
| MERS-CoV | MASPAAPRAVSFADNNDITNTNLSRGRGRN |
| Nucleocapsid | PKPRAAPNNTVSWYTGLTQHGKVPLTFPPG |
| protein (SEQ | QGVPLNANSTPAQNAGYWRRQDRKINTGNG |
| ID NO: 15) | IKQLAPRWYFYYTGTGPEAALPFRAVKDGI |
| | VWVHEDGATDAPSTFGTRNPNNDSAIVTQF |
| | APGTKLPKNFHIEGTGGNSQSSSRASSLSR |
| | NSSRSSSQGSRSGNSTRGTSPGPSGIGAVG |
| | GDLLYLDLLNRLQALESGKVKQSQPKVITK |
| | KDAAAAKNKMRHKRTSTKSFNMVQAFGLRG |
| | PGDLQGNFGDLQLNKLGTEDPRWPQIAELA |
| | PTASAFMGMSQFKLTHQNNDDHGNPVYFLR |
| | YSGAIKLDPKNPNYNKWLELLEQNIDAYKT |
| | FPKKEKKQKAPKEESTDQMSEPPKEQRVQG |
| | SITQRTRTRPSVQPGPMIDVNTD |

Examples of fungus include, but are not limited to: Cryptococcus species including Crytococcus neoformans, Histoplasma species including Histoplasma capsulatum, Coccidioides species including Coccidiodes immitis, Paracoccidioides species including Paracoccidioides brasiliensis, Blastomyces species including Blastomyces dermatitidis, Candida species including Candida albicans, Sporothrix species including Sporothrix schenckcii, Aspergillus species, and fungi of mucormycosis. In some embodiments, the fungus is Candida spp., Aspergillus spp., Cryptococcus spp., Mucormycete, Blastomyces dermatitidis (causing blastomycosis), or endemic mycosis causing fungus such as Histoplasma capsulatum (causing histoplasmosis), or Sporothrix schenckii (causing sporotrichosis).

Other infectious organisms include, without limitation: parasites. Parasites include Plasmodium species, such as Plasmodium species including Plasmodium ilciparum, Plasmodium malariae, Plasmodium ovale, Plasmodium vivax, Toxoplasma gondii. Blood-borne and/or tissue-borne parasites include Plasmodium species, Babesia species including Babesia microti and Babesia divergens, Leishmania species including Leishmania tropica, Leishmania species, Leishmania braziliensis, Leishmania donovani, Trypanosoma species including Trypanosoma gambiense, Trypanosoma rhodesiense (African sleeping sickness), and Trypanosoma cruzi (Chagas' disease). In some embodiments, the parasite is a Plasmodium spp., Leishmania, or a helminth.

Other medically relevant microorganisms have been described extensively in the literature, e.g., see C. G. A Thomas, Medical Microbiology, Bailliere Tindall, Great Britain 1983, incorporated herein by reference.

In some embodiments, an antigen of the present disclosure comprises a cancer-specific antigen and/or a nucleic acid encoding such. A “cancer-specific antigen” refers to a protein that is specifically expressed or upregulated in a cancer cell, as compared to non-cancerous cells of the same origin. A cancer-specific antigen, or an epitope derived thereof, can be recognized by the immune system to induce an immune response against the cancer. Classes of proteins that may be cancer-specific antigen include, without limitation: enzymes, receptors, and transcription factors.

A large number of proteins that are specifically expressed in cancer cells or are upregulated in cancer cells have been identified (Hassane et al., Holland-Frei Cancer Medicine. 6th Edition, incorporated herein by reference). Known cancer-specific antigens are classified into different classes: cancer/testis antigens (e.g., MAGE family members or NY-ESO-1), differentiation antigens (e.g., tyrosinase and Melan-A/MART-1 for melanoma, and PSA for prostate cancer), overexpressed cancer-specific antigens (e.g., Her-2/neu, Survivin, Telomerase and WT1), cancer-specific antigens arising from mutations of normal genes (e.g., mutated 3-catenin or CDK4), cancer-specific antigens arising from abnormal post-translational modifications (e.g., altered glycosylation patterns) that lead to novel epitopes in tumors (e.g., MUC1), and oncoviral proteins (e.g., human papilloma type 16 virus proteins, E6 and E7). In some embodiments, the tumor-specific antigen is expressed in a broad range of different types of cancers. In some embodiments, the tumor-specific antigen is expressed only in one or relatively few types of cancer.

In some embodiments, an antigen comprises a fragment or an epitope derived from a cancer-specific antigen and/or a nucleic acid encoding such. For example, the fragment or an epitope derived from a cancer-specific antigen may be 5-40 amino acids long. In some embodiments, the fragment or an epitope derived from a cancer-specific antigen is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids long.

In some embodiments, a fragment or epitope derived from a cancer-specific antigen is a heteroclitic epitope. A “heteroclitic epitope” refers to an altered version of an endogenous peptide sequence (i.e., an analog) from a cancer-specific antigen engineered to elicit potent immune reactions. Heteroclitic epitopes have increased stimulatory capacity or potency for a specific T cell, as measured by an increased response to a given dose, or by requiring a lesser amount of the epitope to achieve the same response. Heteroclitic epitopes may therefore be beneficial as vaccine components since these epitopes induce T cell responses stronger than those induced by the native epitope.

In some embodiments, a heteroclitic epitope comprises modifications, e.g., amino acid substitutions, as compared to the native sequence in the cancer-specific antigen. In some embodiments, the heteroclitic epitope comprises more than one amino acid substitution (e.g., 2, 3, 4, 5, or more substitutions) compared to the native sequence of the cancer-specific antigen it is derived from. In some embodiments, a heteroclitic epitope is at least 60%, at least 70%, at least 80%, at least 90%, at least 98%, or at least 99% identical to the native sequence that it is derived from. In some embodiments, a heteroclitic epitope is 60%, 65%, 70%, 75%, 80%, 85%, 90%, 95%, 98%, or 99% identical to the native sequence that it is derived from.

In some embodiments, a heteroclitic epitope is more immunogenic than a peptide of its native sequence. For example, a heteroclitic epitope may be at least 30% more immunogenic (i.e., induces a stronger immune response) than its corresponding native peptide. In some embodiments, a heteroclitic epitope may be at least 30%, at least 40%, at least 50%, at least 60%, at least 70%, at least 80%, at least 90%, at least 100%, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, or more immunogenic than its corresponding native peptide.

In some embodiments, the fragment or epitope derived from a cancer-specific antigen is a cryptic epitope. A “cryptic epitope” refers to an epitope derived from a cancer-specific antigen that does not necessarily undergo antigen processing/presentation and are therefore ‘hidden’ from immune recognition. Cryptic epitopes usually appear in very low concentration on antigen presenting cells (APC) and do not elicit the removal of auto-reactive T cells. Cryptic epitopes are not presented for recognition by T cells unless they are produced in unusually large concentrations or unless they are freed from the configuration of their native antigen. Cryptic epitopes derived from cancer-specific antigens may be used to break the tolerance of T cells to the tumor and induce potent immune response against the tumor. Such principles have been described in Pardoll, et al., PNAS, Vol. 96, pp. 5340-5342 (1999), the entire contents of which are incorporated herein by reference.

In some embodiments, a cryptic epitope is generated from translation of a non-coding region of the cancer-specific antigen gene or translation of a different reading frame of a coding region of the cancer-specific antigen. A cryptic epitope may be more immunogenic (i.e., induces a stronger immune response) than any native peptide derived from the cancer-specific antigen. For example, a cryptic epitope may be at least 30% more immunogenic than any native peptide derived from the cancer-specific antigen. In some embodiments, a cryptic epitope is at least 30%, at least 40%, at least 50%, at least 60%, at least 70%, at least 80%, at least 90%, at least 100%, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, or more immunogenic than any native peptide derived from the cancer-specific antigen. One skilled in the art is familiar with how to assess the immune response induced by an antigen, e.g., measuring antibody titers.

In some embodiments, a cancer-specific antigen is a neoantigen. A “neoantigen” refers to an antigen generated via random somatic mutations occurring in cancer cells and are thus specific to the lineage of the cancer cells it is derived from. Neoantigens are regarded in the art to be responsible for the immunogenicity of tumors (Srivastava et al., 1993, Duan et al., 2009; van der Bruggen et al., 2013, incorporated herein by reference), and mathematic modeling has predicted the existence of tens to hundreds of neoepitopes (epitopes derived from neoantigens) in individual human tumors (Srivastava 2009, incorporated herein by reference). The recent revolution in high-throughput DNA sequencing and accompanying bioinformatics approaches has finally made it possible to actually identify the individually specific neoepitopes in individual cancers.

In some embodiments, an antigen described herein is an antigen designed to provide broad heterologous protection against a range of pathogens. Heterologous immunity refers to the phenomenon whereby a history of an immune response against a stimulus or pathogen can provide a level of immunity to a second unrelated stimulus or pathogen (e.g., as described in Chen et al., Virology 2015 482: 89-97, incorporated herein by reference). For example, an antigen that induces cross-reactive memory CD8+ T cells against multiple unrelated viruses such as influenza A and Epstein-Barr Virus (EBV), as described in Watkin et al., J Allerg Clin Immunol 2017 October; 140(4) 1206-1210, incorporated herein by reference. In some embodiments, the compound of Formula (I) induces and/or enhances heterologous protection.

Polypeptide or polynucleotide molecules of the present disclosure may share a certain degree of sequence similarity or identity with reference molecules (e.g., reference polypeptides or reference polynucleotides), for example, wild-type molecules. The term “identity” as known in the art, refers to a relationship between the sequences of two or more polypeptides or polynucleotides, as determined by comparing the sequences. In the art, identity also means the degree of sequence relatedness between them as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197.) A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453.). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g., between nucleic acid molecules (e.g., DNA molecules and/or RNA molecules) and/or between polypeptide molecules. Polymeric molecules (e.g., nucleic acid molecules (e.g., DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids.

In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function over the course of evolution, whereas paralogs can evolve new functions, even if these are related to the original one.

The term “identity” refers to the overall relatedness between polymeric molecules, for example, between polynucleotide molecules (e.g., DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In some embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

### Compositions and Vaccines

In some embodiments, compositions (e.g., pharmaceutical composition) of the present disclosure comprise the compound of Formula (I). In some embodiments, the composition comprises the compound of Formula (I) and an antigen. As contemplated herein, the terms “composition” and “formulation” may be used interchangeably.

In some embodiments, the compound of Formula (I) is conjugated to the antigen. In some embodiments, the compound of Formula (I) is not conjugated to the antigen. In some embodiments, the compound of Formula (I) is lipidated.

Methods of conjugating a compound to another molecule (e.g., a protein or a nucleic acid) are known to those skilled in the art. For example, in some embodiments, conjugation may be achieved via reactive chemical groups by incorporating one of a pair of chemical groups that react with one another to each of the two molecules to be conjugated. A “reactive chemical group” or “functional chemical group” refers to specific groups (moieties) of atoms or bonds within molecules that are responsible for the characteristic chemical reactions of those molecules. These terms are used interchangeably herein. One example of such reactive group is a “click chemistry handle.” Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “Click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgon cycloaddition; thiol-yne addition; imine formation; and Michael additions (e.g., maleimide addition). Non-limiting examples of a click chemistry handle include an azide handle, an alkyne handle, or an aziridine handle. Azide is the anion with the formula N₃⁻. It is the conjugate base of hydrazoic acid (HN₃). N₃⁻ is a linear anion that is isoelectronic with CO₂, NCO⁻, N₂O, NO₂⁺ and NCF. Azide can be described by several resonance structures, an important one being N⁻═N⁺═N⁻. An alkyne is an unsaturated hydrocarbon containing at least one carbon-carbon triple bond. The simplest acyclic alkynes with only one triple bond and no other functional groups form a homologous series with the general chemical formula CₙH₂ₙ₋₂. Alkynes are traditionally known as acetylenes, although the name acetylene also refers specifically to C₂H₂, known formally as ethyne using IUPAC nomenclature. Like other hydrocarbons, alkynes are generally hydrophobic but tend to be more reactive. Aziridines are organic compounds containing the aziridine functional group, a three-membered heterocycle with one amine group (—NH—) and two methylene bridges (—CH₂—). The parent compound is aziridine (or ethylene imine), with molecular formula C₂H₅N.

Other non-limiting, exemplary reactive groups include: acetals, ketals, hemiacetals, and hemiketals, carboxylic acids, strong non-oxidizing acids, strong oxidizing acids, weak acids, acrylates and acrylic acids, acyl halides, sulfonyl halides, chloroformates, alcohols and polyols, aldehydes, alkynes with or without acetylenic hydrogen amides and imides, amines, aromatic, amines, phosphines, pyridines, anhydrides, aryl halides, azo, diazo, azido, hydrazine, and azide compounds, strong bases, weak bases, carbamates, carbonate salts, chlorosilanes, conjugated dienes, cyanides, inorganic, diazonium salts, epoxides, esters, sulfate esters, phosphate esters, thiophosphate esters borate esters, ethers, soluble fluoride salts, fluorinated organic compounds, halogenated organic compounds, halogenating agents, aliphatic saturated hydrocarbons, aliphatic unsaturated hydrocarbons, hydrocarbons, aromatic, insufficient information for classification, isocyanates and isothiocyanates, ketones, metal hydrides, metal alkyls, metal aryls, and silanes, alkali metals, nitrate and nitrite compounds, inorganic, nitrides, phosphides, carbides, and silicides, nitriles, nitro, nitroso, nitrate, nitrite compounds, organic, non-redox-active inorganic compounds, organometallics, oximes, peroxides, organic, phenolic salts, phenols and cresols, polymerizable compounds, quaternary ammonium and phosphonium salts, strong reducing agents, weak reducing agents, acidic salts, basic salts, siloxanes, inorganic sulfides, organic sulfides, sulfite and thiosulfate salts, sulfonates, phosphonates, organic thiophosphonates, thiocarbamate esters and salts, and dithiocarbamate esters and salts. In some embodiments, the reactive group is a carboxylic acid group.

The compositions comprising the compound of Formula (I) or the compound of Formula (I) and an antigen described herein are immunogenic. Being “immunogenic” means that the composition elicits an immune response when administered to a subject (e.g., a mammalian subject such as a human). As used herein, an “immune response” refers to a response by a cell of the immune system, such as an antigen-presenting cell, dendritic cell, monocyte, macrophage, NKT cell, NK cell, basophil, eosinophil, or neutrophil, B cell, T cell (CD4+ or CD8+), regulatory T cell, antigen-presenting cell, dendritic cell, monocyte, macrophage, NKT cell, NK cell, basophil, eosinophil, or neutrophil, to a stimulus (e.g., to an antigen or an adjuvant).

In some embodiments, the immune response elicited by the composition described herein is specific for a particular antigen (an “antigen-specific response” or “adaptive immune response”) and refers to a response by a CD4+ T cell, a CD8+ T cell, or a B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

In some embodiments, an antigen-specific immune response includes both a humoral and/or a cell-mediated immune response to the antigen. A “humoral immune response” is an antibody-mediated immune response and involves the induction and generation of antibodies that recognize and bind with some affinity for the antigen in the immunogenic composition of the invention, while a “cell-mediated immune response” is one mediated by T-cells and/or other white blood cells. A “humoral immune response” may alternately be referred to as a “mucosal immune response”. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+T helper cells or CD8+ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

In some embodiments, the immune response elicited by the composition described herein is an innate immune response. An “innate immune response” refers to the response by the innate immune system. The innate immune system uses a set of germline-encoded receptors (“pattern recognition receptor” or “PRR”) for the recognition of conserved molecular patterns present in microorganisms. These molecular patterns occur in certain constituents of microorganisms including: lipopolysaccharides, peptidoglycans, lipoteichoic acids, phosphatidyl cholines, bacteria-specific proteins, including lipoproteins, bacterial DNAs, viral single and double-stranded RNAs, unmethylated CpG-DNAs, mannans and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism. In some embodiments, the innate immune response elicited by the composition described herein confers heterologous (“non-specific”) immunity to a broad range of pathogenic microbes by enhancing innate immune responses to subsequent stimuli, a phenomenon known as “trained immunity”, a form of innate memory, e.g., as described in Netea et al. (Trained Immunity: An Ancient Way of Remembering. Cell Host Microbe. 2017 Mar. 8; 21(3):297-300, incorporated herein by reference).

The receptors of the innate immune system that recognize PAMPs are called Pattern Recognition Receptors (PRRs). (Janeway et al. (1989) Cold Spring Harb. Symp. Quant. Biol. 54: 1-13; Medzhitov et al. (1997) Curr. Opin. Immunol. 94: 4-9, incorporated herein by reference). PRRs vary in structure and belong to several different protein families. Some of these receptors recognize PAMPs directly (e.g., CD14, DEC205, collectins), while others (e.g., complement receptors) recognize the products generated by PAMP recognition. Members of these receptor families can, generally, be divided into three types: 1) humoral receptors circulating in the plasma; 2) endocytic receptors expressed on immune-cell surfaces, and 3) signaling receptors that can be expressed either on the cell surface or intracellularly. (Medzhitov et al. (1997) Curr. Opin. Immunol. 94: 4-9; Fearon et al. (1996) Science 272: 50-3, incorporated herein by reference). Non-limiting examples of PRRs include: Toll-like receptors (e.g., TLR2, TLR4, TLR7, TLR8, TLR9), NOD1/2, RIG-1/MDA-5, C-type lectins, and STING. Cellular PRRs are expressed on or within effector cells of the innate immune system, including cells that function as professional antigen-presenting cells (APC) in adaptive immunity. Such effector cells include, but are not limited to, macrophages, dendritic cells, B lymphocytes and surface epithelia. This expression profile allows PRRs to directly induce innate effector mechanisms, and also to alert the host organism to the presence of infectious agents by inducing the expression of a set of endogenous signals, such as inflammatory cytokines and chemokines, including, without limitation: chemokines, interferons, interleukins, lymphokines, and tumor necrosis factors. This latter function allows efficient mobilization of effector forces to combat pathogens or cancer cells.

In some embodiments, immunogenic compositions comprising the compound of Formula (I) described herein elicit an innate immune response by activating a Toll-like receptor (TLR), such as, but not limited to, TLR8. In some embodiments, the compound of Formula (I) in such a composition elicits an innate immune response by activating TLR8. Without wishing to be bound by theory, TLR8 (also referred to as cluster of differentiation 288; CD288) is a highly conserved Toll-like receptor that is localized to endosomal membranes within APCs, such as phagocytes. TLR8 is critical for mediating innate and adaptive immune responses against nucleic acids produced by pathogens, particularly single-stranded RNA species produced by certain viruses and bacteria. TLR8 recognizes GU-rich single stranded RNA in particular, including those derived from HIV-1 and Beta coronaviruses, such as SARS-CoV-1 and SARS-CoV-2 (see, e.g., Campbell G R, et al. “SARS-CoV-2, SARS-CoV-1, and HIV-1 derived ssRNA sequences activate the NLRP3 inflammasome in human macrophages through a non-classical pathway”. iScience. 2021; 24(4):102295, which is incorporated herein by reference). Upon binding with ssRNA, TLR8 dimerizes, bringing Toll/interleukin-1 receptor/resistance protein (TIR) domains of the dimer into contact and leading to the subsequent recruitment of the adaptor protein MyD88. Through interleukin-1 receptor associated kinase (IRAK), MyD88 activates a signaling cascade which leads to the activation of the transcription factor NF-κB, which subsequently stimulates the expression of numerous inflammatory cytokines, chemokines, and additional proteins and peptides that induce inflammation and stimulate adaptive immunity.

In some embodiments, compositions comprising the compound of Formula (I) induce a T cell helper 1 (Th1) immune response. A “Th1 immune response” refers to an immune response that is characterized by the preferential polarization (differentiation) of cytokine-secreting T cells into Th1 cells, rather than Th2 cells. Th1 and Th2 cells have cytokine expression profiles that are generally not overlapping and lead to different types of immune responses (Berger A, BMJ. 2000; 321(7258):424, which is incorporated herein by reference). Th1 cells primarily express and secrete cytokines that lead to an inflammatory response, such as IFNγ. In contrast, Th2 cells tend to express and secrete non-inflammatory cytokines, such as IL-4, IL-5, and IL-13, which are associated with the production of IgE as well as the activity of eosinophils. Th2 responses are closely linked to allergic reactions. Th2 responses also include expression of anti-inflammatory cytokines, such as IL-10, and are generally regarded to counteract Th1 immune responses.

In some embodiments, compositions comprising the compound of Formula (I) induce the production of one or more signaling molecules such as, but not limited to, a cytokine or a chemokine. A composition comprising the compound of Formula (I) may, for example, induce the production of one or more cytokines or chemokines that are proinflammatory (capable of inducing inflammation) or Th-polarizing (capable of inducing polarization of Th1 or Th2 cells). It is demonstrated herein that the compound of Formula (I) alone is sufficient to induce cytokine (e.g., TNF, IL-6, IL-10, IL-12 p40, IL-12 p70, GM-CSF, IFN-γ, IFN-α2, IL1-β, IP-10) and/or chemokine (e.g., CCL2 (MCP1), CCL3 (MIP1α), CCL5 (RANTES), CXCL8 (IL-8)) production by human peripheral blood mononuclear cells (PBMC) in vitro and enhanced antigen-specific immune response against SARS-CoV-2 spike protein antigen in vivo.

In some embodiments, the composition comprising the compound of Formula (I) and an antigen is a vaccine composition. A “vaccine composition” is a composition that activates or enhances a subject's immune response to an antigen after the vaccine is administered to the subject. In some embodiments, a vaccine stimulates the subject's immune system to recognize the antigen as foreign and enhances the subject's immune response if the subject is later exposed to a particular pathogen, whether attenuated, inactivated, killed, or not. Vaccines may be prophylactic, for example, preventing or ameliorating a detrimental effect of a future exposure to a pathogen, or therapeutic, for example, activating the subject's immune response to a pathogen after the subject has been exposed to the pathogen. In some embodiments, a vaccine composition is used to protect or treat an organism against a disease (e.g., an infectious disease or cancer). In some embodiments, the vaccine is a subunit vaccine (e.g., a recombinant subunit vaccine), an attenuated vaccine (e.g., containing an attenuated pathogen such as a bacterial cell or a viral genome), a live vaccine (e.g., containing a live attenuated pathogen such as a bacterium or virus), or a conjugated vaccine (e.g., a vaccine containing an antigen that is not very immunogenic covalently attached to an antigen that is more immunogenic). One non-limiting example of a conjugated vaccine comprises a LPS attached to a strong protein antigen.

The terms “vaccine composition” and “vaccine” are used interchangeably herein. Vaccines that contain cancer-specific antigens are termed herein as “cancer vaccine.” Cancer vaccines induce cancer-specific immune response against a cancer or a cancer-specific antigen. Such an immune response is effective in inhibiting cancer growth and/or preventing reoccurrence of a cancer and/or tumor. Cancer vaccines may be used for cancer immunotherapy, which is a type of cancer treatment designed to boost the body's natural defenses to fight the cancer. It uses substances either made by the body or in a laboratory to improve or restore immune system function.

An “adjuvant” refers to a pharmacological or immunological agent that modifies the effect of other agents, for example, of an antigen in a vaccine. Adjuvants are typically included in vaccines to enhance the recipient subject's immune response to an antigen. The use of adjuvants allows the induction of a greater immune response in a subject with the same dose of antigen, or the induction of a similar level of immune response with a lower dose of injected antigen.

Adjuvants are thought to function in several ways, including by increasing the surface area of antigen, prolonging the retention of the antigen in the body thus allowing time for the lymphoid system to have access to the antigen, slowing the release of antigen, targeting antigen to macrophages, activating macrophages, activating leukocytes such as antigen-presenting cells (e.g., monocytes, macrophages, and/or dendritic cells), or otherwise eliciting broad activation of the cells of the immune system see, e.g., H. S. Warren et al, Annu. Rev. Immunol., 4:369 (1986), incorporated herein by reference. The ability of an adjuvant to induce and increase a specific type of immune response and the identification of that ability is thus a key factor in the selection of particular adjuvants for vaccine use against a particular pathogen. Adjuvants that are known to those of skill in the art, include, without limitation: aluminum salts (referred to herein as “alum”), liposomes, lipopolysaccharide (LPS) or derivatives such as monophosphoryl lipid A (MPLA) and glycopyranosyl lipid A (GLA), molecular cages for antigen, components of bacterial cell walls, endocytosed nucleic acids such as double-stranded RNA (dsRNA), single-stranded DNA (ssDNA), and unmethylated CpG dinucleotide-containing DNA. Typical adjuvants include water and oil emulsions, e.g., Freund's adjuvant and MF59, and chemical compounds such as aluminum hydroxide or alum. At present, currently licensed vaccines in the United States contain only a limited number of adjuvants, such as alum that enhances production of subtype 2 helper T (Th2) cells and MPLA which activates innate immunity via Toll-like receptor 4 (TLR4). Many of the most effective adjuvants include bacteria or their products, e.g., microorganisms such as the attenuated strain of Mycobacterium bovis, Bacille Calmette-Guérin (BCG); microorganism components, e.g., alum-precipitated diphtheria toxoid, bacterial lipopolysaccharides (“endotoxins”) and their derivatives such as MPLA and GLA.

In some embodiments, the vaccine composition described herein further comprises a second adjuvant, in addition to the compound of Formula (I) as the first adjuvant. Any adjuvants known in the art or described herein may be used as the second adjuvant in the composition. In some embodiments, the second adjuvant is an agonist of a Pattern Recognition Receptor (PRR) such as a Toll-like receptor (TLR), a NOD-like receptor (NLR), a RIG-I-like receptor (RLR), a C-type Lectin receptor (CLRs), or a stimulator of interferon genes (STING). An “agonist” is a chemical that binds to a receptor and activates the receptor to produce a biological response. Agonists of the PPRs enhance immune responses (e.g., innate or adaptive immune response). Agonists of PPRs are known to those skilled in the art. For example, various TLR and NLR agonists are described in Kaczanowska et al, J Leukoc Biol. 2013 June; 93(6): 847-863; Higgins et al., Curr Infect Dis Rep. 2010 January; 12(1):4-12; and Maisonneuve et al., Proc Natl Acad Sci USA. 2014 Aug. 26; 111(34): 12294-12299, incorporated herein by reference. RIG-I-like receptor agonists are described in Ranjith-Kumar et al., J Biol Chem. 2009-1-9; 284(2): 1155-1165; and Goulet et al., PLOS Pathogens 9(8): 10, incorporated herein by reference. CLR agonists are described in Lamb et al., Biochemistry. 2002 Dec. 3; 41(48):14340-7; and Yan et al., Front Immunol. 2015; 6: 408, incorporated herein by reference. STING agonists are described in Fu et al., Sci Transl Med. 2015 Apr. 15; 7(283): 283ra52; and Foote et al., Cancer Immunology Research, DOI: 10.1158/2326-6066.CIR-16-0284, incorporated herein by reference. The PRR agonists described herein are also commercially available, e.g., from InvivoGen (California, USA). In some embodiments, the second adjuvant is alum.

In some embodiments, a vaccine composition described herein is formulated for administration to a subject. In some embodiments, the vaccine composition is formulated or administered in combination with one or more pharmaceutically acceptable excipients. In some embodiments, vaccine compositions comprise at least one additional active substance, such as, for example, a therapeutically active substance, a prophylactically active substance, or a combination of both. Vaccine compositions may be sterile, pyrogen-free, or both. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

Formulations of the vaccine compositions described herein may be prepared by any method known or hereafter developed in the art of pharmacology. In general, such preparatory methods include the step of bringing the antigen and/or the adjuvant (e.g., the compound of Formula (I)) into association with an excipient and/or one or more other accessory ingredients, and then, if necessary and/or desirable, dividing, shaping and/or packaging the product into a desired single- or multi-dose unit.

Relative amounts of the antigen, the adjuvant, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, a vaccine composition described herein is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with DNA or RNA vaccines (e.g., for transplantation into a subject), hyaluronidase, nanoparticle mimics and combinations thereof.

In some embodiments, a vaccine composition is formulated in an aqueous solution. In some embodiments, the vaccine composition is formulated in a nanoparticle. In some embodiments, the vaccine composition is formulated in a lipid nanoparticle. In some embodiments, the vaccine composition is formulated in a lipid-polycation complex, referred to as a lipid nanoparticle. In some embodiments, the vaccine composition is formulated in an emulsion such as, for example, an oil-in-water emulsion comprising lipid nanoparticles. The formation of the lipid nanoparticle may be accomplished by methods known in the art and/or as described in U.S. Pub. No. 20120178702, incorporated herein by reference. As a non-limiting example, the polycation may include a cationic peptide or a polypeptide such as, but not limited to, polylysine, polyornithine and/or polyarginine and the cationic peptides described in International Pub. No.

WO2012013326 or US Patent Pub. No. US20130142818; each of which is incorporated herein by reference. In some embodiments, the vaccine composition is formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the ionizable lipid component, the degree of ionizable lipid saturation, the nature of the PEGylation, ratio of all components and biophysical parameters such as size. In one example by Semple et al. (Nature Biotech. 2010 28:172-176; incorporated herein by reference), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200; incorporated herein by reference).

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0% and/or 3.0% to 6.0% of the lipid molar ratio of PEG-c-DOMG (R-3-[(ω-methoxy-poly(ethyleneglycol)2000)carbamoyl)]-1,2-dimyristyloxypropyl-3-amine) (also referred to herein as PEG-DOMG) as compared to the cationic lipid, DSPC and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In some embodiments, a vaccine formulation described herein is a nanoparticle that comprises at least one lipid (termed a “lipid nanoparticle” or “LNP”). The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, incorporated herein by reference. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

Lipid nanoparticle formulations typically comprise a lipid, in particular, an ionizable cationic lipid, for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example a PEG or PEG-modified lipid.

In some embodiments, a lipid nanoparticle formulation consists essentially of (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of 20-60% ionizable cationic lipid: 5-25% neutral lipid: 25-55% sterol; 0.5-15% PEG-lipid.

Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51: 8529-8533; and Maier et al. (2013) Molecular Therapy 21, 1570-1578 (the contents of each of which are incorporated herein by reference in their entirety).

The lipid nanoparticles described herein may be made in a sterile environment by the system and/or methods described in US Patent Publication No. US20130164400, incorporated herein by reference.

In some embodiments, a lipid nanoparticle formulation may be formulated by the methods described in International Publication Nos. WO2011127255 or WO2008103276, the contents of each of which are herein incorporated by reference in their entirety. As a non-limiting example, the antigen and the compound of Formula (I) described herein may be encapsulated in LNP formulations as described in WO2011127255 and/or WO2008103276; the contents of each of which are herein incorporated by reference in their entirety.

In some embodiments, lipid nanoparticle formulations described herein may comprise a polycationic composition. As a non-limiting example, the polycationic composition may be selected from formula 1-60 of US Patent Publication No. US20050222064; the content of which is incorporated herein by reference. In another embodiment, the LNP formulations comprising a polycationic composition may be used for the delivery of the modified RNA described herein in vivo and/or in vitro.

In some embodiments, the lipid nanoparticle formulations described herein may additionally comprise a permeability enhancer molecule. Non-limiting permeability enhancer molecules are described in US Patent Publication No. US20050222064; the content of which is incorporated herein by reference.

In some embodiments, the vaccine compositions may be formulated in liposomes such as, but not limited to, DiLa2 liposomes (Marina Biotech, Bothell, WA), SMARTICLES® (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5(12) 1708-1713); incorporated herein by reference) and hyaluronan-coated liposomes (Quiet Therapeutics, Israel).

In some embodiments, the vaccine compositions may be formulated in a lyophilized gel-phase liposomal composition as described in US Publication No. US2012060293, incorporated herein by reference.

In some embodiments, the vaccine compositions described herein may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm, and/or about 90 to about 100 nm.

In some embodiments, the lipid nanoparticles may have a diameter from about 10 to 500 nm. In some embodiments, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm, or greater than 1000 nm.

In some embodiments, the vaccine composition is formulated in a liposome. Liposomes are artificially prepared vesicles which may primarily be composed of a lipid bilayer and may be used as a delivery vehicle for the administration of nutrients and pharmaceutical formulations.

Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No.

US20130177638, US20130177637, US20130177636, US20130177635, US20130177634, US20130177633, US20130183375, US20130183373 and US20130183372, the contents of each of which are incorporated herein by reference.

In some embodiments, the vaccine compositions described herein may include, without limitation, liposomes such as those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; incorporated herein by reference) and liposomes which may deliver small molecule drugs such as, but not limited to, DOXIL® from Janssen Biotech, Inc. (Horsham, PA).

In some embodiments, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method

In some embodiments, liposomes may be formulated for targeted delivery. As a non-limiting example, the liposome may be formulated for targeted delivery to the liver. The liposome used for targeted delivery may include, but is not limited to, the liposomes described in and methods of making liposomes described in US Patent Publication No. US20130195967, the contents of which are incorporated herein by reference.

In some embodiments, the antigen and/or the compound of Formula (I) may be formulated in a cationic oil-in-water emulsion where the emulsion particle comprises an oil core and a cationic lipid which can interact with the polynucleotide anchoring the molecule to the emulsion particle (see International Pub. No. WO2012006380; incorporated herein by reference).

In some embodiments, the antigen and/or the compound of Formula (I) may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are incorporated herein by reference.

The antigen, the compound of Formula (I), and/or optionally the second adjuvant may be formulated using any of the methods described herein or known in the art separately or together. For example, the antigen and compound of Formula (I) may be formulated in one lipid nanoparticle or two separately lipid nanoparticles. In some embodiments, the antigen and the compound of Formula (I) are formulated in the same aqueous solution or two separate aqueous solutions. In some embodiments, the antigen and the compound of Formula (I), and/or optionally the second adjuvant is adsorbed onto alum (e.g., as described in Jones et al., Journal of Biological Chemistry 280, 13406-13414, 2005, incorporated herein by reference).

In some embodiments, the vaccine composition described herein comprises two or more adjuvants (also referred to as an “adjuvant system”). The adjuvant system comprises the compound of Formula (I) and one or more other adjuvants described herein.

### Methods for Enhancing an Immune Response

Other aspects of the present disclosure provide methods of enhancing an immune response in a subject. In some embodiments, the methods comprise administering to the subject an effective amount of the compound of Formula (I) described herein (e.g., for enhancing an innate immune response, including induction of heterologous or “trained” immunity or innate memory). In some embodiments, the methods comprise administering to the subject an effective amount of the compound of Formula (I) and an effective amount of an antigen (e.g., for enhancing an antigen-specific immune response). In some embodiments, the methods comprise administering to the subject an effective (e.g., therapeutically effective) amount of the compound of Formula (I). In some embodiments, the compound of Formula (I) is administered separately from the antigen. In some embodiments, the compound of Formula (I) is administered prior to administering the antigen. In some embodiments, the compound of Formula (I) is administered after administering the antigen. In some embodiments, the compound of Formula (I) and the antigen are administered simultaneously. In some embodiment, the compound of Formula (I) and the antigen are administered as an admixture.

The antigen and/or the compound of Formula (I) (e.g., the antigen alone, the compound of Formula (I) alone, or the antigen and the compound of Formula (I) together) described herein elicits an immune response in the subject. In some embodiments, the antigen and/or the compound of Formula (I) elicits a Th1 immune response in the subject. In some embodiments, the antigen and/or the compound of Formula (I) activates cytokine (e.g., TNF, IL-6, IL-10, IL-12 p40, IL-12 p70, GM-CSF, IFN-γ, IFN-α2, IL1-β, IP-10) and/or chemokine (e.g., CCL2 (MCP1), CCL3 (MIP1α), CCL5 (RANTES), CXCL8 (IL-8)) production. In some embodiments, the immune response is an innate immune response. In some embodiments, the immune response is an adaptive immune response specific to the antigen in the composition or vaccine. In some embodiments, the antigen and/or the compound of Formula (I) activates B cell immunity. In some embodiments, the antigen and/or the compound of Formula (I) elicits antibody production. In some embodiments, the composition or the vaccine activates cytotoxic T cells specific to the antigen.

In some embodiments, the compound of Formula (I), whether administered alone or in an admixture with an antigen, enhances the innate immune response, compared to the absence of the compound of Formula (I) or when the antigen is administered alone. In some embodiments, the compound of Formula (I) activates peripheral blood mononuclear cells (PBMCs). In some embodiments, the number of PBMCs that are activated is increased by at least 20% in the presence of the compound of Formula (I), compared to the absence of the compound of Formula (I) or when the antigen is administered alone. For example, the number of PBMCs that are activated may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the compound of Formula (I), compared to the absence of the compound of Formula (I) or when the antigen is administered alone. In some embodiments, the number of PBMCs that are activated is increased by 20%, 30%, 40%, 50%, 60%, 70%, 80%, 90%, 100%, 2-fold, 5-fold, 10-fold, 100-fold, 1000-fold or more, in the presence of the compound of Formula (I), compared to the absence of the compound of Formula (I) or when the antigen is administered alone.

In some embodiments, the compound of Formula (I) activates a pattern recognition receptor (PRR). In some embodiments, the PRR is selected from the group consisting of Toll-like receptors (e.g., TLR8). In some embodiments, the PPR activation is increased by at least 20% in the presence the compound of Formula (I), compared to the absence of the compound of Formula (I) or when the antigen is administered alone. For example, PPR activation may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the compound of Formula (I), compared to the absence of the compound of Formula (I) or when the antigen is administered alone. In some embodiments, the number of PRRs that are activated is increased by 20%, 30%, 40%, 50%, 60%, 70%, 80%, 90%, 100%, 2-fold, 5-fold, 10-fold, 100-fold, 1000-fold or more, in the presence of the compound of Formula (I), compared to the absence of the compound of Formula (I) or when the antigen is administered alone.

In some embodiments, the compound of Formula (I) induces the production of one or more signaling molecules, such as a cytokine (e.g., TNF, IL-6, IL-10, IL-12 p40, IL-12 p70, GM-CSF, IFN-γ, IFN-α2, IL1-β, IP-10) and/or a chemokine (e.g., CCL2 (MCP1), CCL3 (MIP1α), CCL5 (RANTES), CXCL8 (IL-8)) in the subject. In some embodiments, the level of cytokines (e.g., TNF, IL-6, IL-10, IL-12 p40, IL-12 p70, GM-CSF, IFN-γ, IFN-α2, IL1-β, IP-10) and/or chemokines (e.g., CCL2 (MCP1), CCL3 (MIP1α), CCL5 (RANTES), CXCL8 (IL-8)) is increased by at least 20% in the presence of the compound of Formula (I), compared to the absence of the compound of Formula (I) or when the antigen is administered alone. For example, the level of cytokines (e.g., TNF, IL-6, IL-10, IL-12 p40, IL-12 p70, GM-CSF, IFN-γ, IFN-α2, IL1-β, IP-10) and/or chemokines (e.g., CCL2 (MCP1), CCL3 (MIP1α), CCL5 (RANTES), CXCL8 (IL-8)) may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the compound of Formula (I), compared to the absence of the compound of Formula (I) or when the antigen is administered alone. In some embodiments, the level of cytokines (e.g., TNF, IL-6, IL-10, IL-12 p40, IL-12 p70, GM-CSF, IFN-γ, IFN-α2, IL1-β, IP-10) and/or chemokines (e.g., CCL2 (MCP1), CCL3 (MIP1α), CCL5 (RANTES), CXCL8 (IL-8)) is increased by 20%, 30%, 40%, 50%, 60%, 70%, 80%, 90%, 100%, 2-fold, 5-fold, 10-fold, 100-fold, 1000-fold or more, in the presence of the compound of Formula (I), compared to the absence of the compound of Formula (I) or when the antigen is administered alone.

In some embodiments, the compound of Formula (I) enhances innate immune memory (also referred to as trained immunity). “Innate immune memory” confers heterologous immunity that provides broad protection against a range of pathogens. In some embodiments, the innate immune memory is increased by at least 20% in the presence of the compound of Formula (I), compared to the absence of the compound of Formula (I) or when the antigen is administered alone. For example, the innate immune memory may be increased by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the presence of the compound of Formula (I), compared to the absence of the compound of Formula (I) or when the antigen is administered alone. In some embodiments, the innate immune memory is increased by 20%, 30%, 40%, 50%, 60%, 70%, 80%, 90%, 100%, 2-fold, 5-fold, 10-fold, 100-fold, 1000-fold or more, in the presence of the compound of Formula (I), compared to without the compound of Formula (I) or when the antigen is administered alone.

In some embodiments, the compound of Formula (I), when administered as an admixture with an antigen (e.g., the vaccine composition described herein), enhances the anti-specific immune response against the antigen or against the invading agent from which the antigen is derived (e.g., a microbial pathogen or cancer), compared the absence of the compound of Formula (I), e.g., when the antigen is administered alone. In some embodiments, the compound of Formula (I) enhances the production of antigen-specific antibodies (i.e., immunoglobulins) in the subject (e.g., by at least 20%), compared to the absence of the compound of Formula (I), e.g., when the antigen is administered alone. For example, the compound of Formula (I) may enhance the production of antigen-specific antibodies (i.e., immunoglobulins) by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more in the subject, compared to the absence of the compound of Formula (I), e.g., when the antigen is administered alone. In some embodiments, the compound of Formula (I) enhances the production of antigen-specific antibodies (i.e., immunoglobulins) by 20%, 30%, 40%, 50%, 60%, 70%, 80%, 90%, 100%, 2-fold, 5-fold, 10-fold, 100-fold, 1000-fold or more, compared the absence of the compound of Formula (I), e.g., when the antigen is administered alone. Antigen-specific antibodies may be of any isotype, such as immunoglobulin A (IgA), immunoglobulin D (IgD), immunoglobulin E (IgE), immunoglobulin G (IgG), or immunoglobulin M (IgM). Antigen-specific antibodies that are IgGs may be those of any subclass, such as IgG1, IgG2, IgG3, or IgG4. One skilled in the art is familiar with how to evaluate the level of an antibody titer, e.g., by enzyme-linked immunosorbent assay (ELISA).

In some embodiments, the compound of Formula (I) enhances the activation of cytotoxic T-cells (e.g., by at least 20%) in the subject, compared to the absence of the compound of Formula (I), e.g., when the antigen is administered alone. For example, the compound of Formula (I) may enhance activation of cytotoxic T-cells by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the subject, compared to the absence of the compound of Formula (I), e.g., when the antigen is administered alone. In some embodiments, the compound of Formula (I) enhances the activation of cytotoxic T-cells by 20%, 30%, 40%, 50%, 60%, 70%, 80%, 90%, 100%, 2-fold, 5-fold, 10-fold, 100-fold, 1000-fold or more, compared to the absence of the compound of Formula (I), e.g., when the antigen is administered alone.

It has been demonstrated that the innate immune system plays a crucial role in the control of initiation of the adaptive immune response and in the induction of appropriate cell effector responses (Fearon et al. (1996) Science 272: 50-3; Medzhitov et al. (1997) Cell 91: 295-8, incorporated herein by reference). As such, in some embodiments, the compound of Formula (I) enhances the innate immune response in a subject (e.g., when administered alone or in an admixture with an antigen), which in turn enhances the adaptive immune response against the antigen in the subject. This is particular useful in subjects that have undeveloped (e.g., in an neonatal infant), weak (e.g., in an elderly), or compromised immune systems (e.g., in a patient with primary immunodeficiency or acquired immunodeficiency secondary to HIV patient infection or a cancer patient undergoing with or without chemotherapy and/or radiation therapy).

In some embodiments, the compound of Formula (I) prolongs the effect of a vaccine (e.g., by at least 20%) in the subject, compared to without the compound of Formula (I) (e.g., when the antigen is administered alone). For example, the compound of Formula (I) may prolong the effect of a vaccine by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more, in the subject, compared to the absence of the compound of Formula (I), e.g., when the antigen is administered alone. In some embodiments, the compound of Formula (I) prolongs the effect of a vaccine by 20%, 30%, 40%, 50%, 60%, 70%, 80%, 90%, 100%, 2-fold, 5-fold, 10-fold, 100-fold, 1000-fold or more, compared to the absence of the compound of Formula (I), e.g., when the antigen is administered alone.

In some embodiments, the compound of Formula (I) increases rate of (accelerates) an immune response, compared to the absence of the compound of Formula (I), e.g., when the antigen is administered alone. For example, the compound of Formula (I) may increase the rate of an immune response by at least 20%, at least 30%, at least 40%, at least 50%, at least 60%, at least 70%, at least 80%, at least 90%, at least 100%, at least 2-fold, at least 5-fold, at least 10-fold, at least 100-fold, at least 1000-fold or more in the subject, compared to the absence of the compound of Formula (I), e.g., when the antigen is administered alone. In some embodiments, the compound of Formula (I) increases the rate of an immune response by 20%, 30%, 40%, 50%, 60%, 70%, 80%, 90%, 100%, 2-fold, 5-fold, 10-fold, 100-fold, 1000-fold or more, compared to the absence of the compound of Formula (I), e.g., when the antigen is administered alone. “Increase the rate of immune response” mean it takes less time for the immune system of a subject to react to an invading agent (e.g., a microbial pathogen).

In some embodiments, the antigen produces a same level of immune response against the antigen at a lower dose in the presence of the compound of Formula (I), compared to the absence of the compound of Formula (I), e.g., when the antigen is administered alone. In some embodiments, the amount of antigen needed to produce the same level of immune response is reduced by at least 20% in the presence of the compound of Formula (I), compared to the absence of the compound of Formula (I), e.g., when the antigen is administered alone. For example, the amount of antigen needed to produce the same level of immune response may be reduced by at least 20%, at least 30%, at least 40%, at least 50%, at least 60%, at least 70%, at least 80%, at least 90%, at least 95%, at least 99% or more, in the presence of the compound of Formula (I), compared to the absence of the compound of Formula (I), e.g., when the antigen is administered alone. In some embodiments, the amount of antigen needed to produce the same level of immune response is reduced by at 20%, 30%, 40%, 50%, 60%, 70%, 80%, 90%, 95%, 99% or more, in the presence of the compound of Formula (I), compared to without the compound of Formula (I), e.g., when the antigen is administered alone.

The prophylactic or therapeutic use of the compound of Formula (I), or the composition or vaccine composition described herein is also within the scope of the present disclosure. In some embodiments, the composition or vaccine composition described herein are used in methods of vaccinating a subject by prophylactically administering to the subject an effective amount of the composition or vaccine composition described herein. “Vaccinating a subject” refers to a process of administering an immunogen, typically an antigen formulated into a vaccine, to the subject in an amount effective to increase or activate an immune response against the antigen and, thus, against a pathogen displaying the antigen. In some embodiments, the terms do not require the creation of complete immunity against the pathogen. In some embodiments, the terms encompass a clinically favorable enhancement of an immune response toward the antigen or pathogen. Methods for immunization, including formulation of a vaccine composition and selection of doses, routes of administration and the schedule of administration (e.g., primary dose and one or more booster doses), are well known in the art. In some embodiments, vaccinating a subject reduces the risk of developing a disease (e.g., an infectious disease or cancer) in a subject.

In some embodiments, a composition or vaccine composition comprising the compound of Formula (I) and optionally an antigen, as described herein, is used in methods of treating a disease (e.g., an infectious disease, allergy, or cancer) by administering to the subject an effective amount of the composition or vaccine composition described herein.

In some embodiments, the disease is an infectious disease. An “infectious disease” refers to an illness caused by a pathogenic biological agent that results from transmission from an infected person, animal, or reservoir to a susceptible host, either directly or indirectly, through an intermediate plant or animal host, vector, or inanimate environment. See Last J M. ed. A dictionary of epidemiology. 4th ed., New York: Oxford University Press, 1988. Infectious disease is also known as transmissible disease or communicable disease. In some embodiments, infectious diseases may be asymptomatic for much or even all of their course in a given host.

Infectious pathogens include some viruses, bacteria, fungi, protozoa, multicellular parasites, and aberrant proteins known as prions. In some embodiments, the infectious disease is caused by any of the microbial pathogens (e.g., a bacterium, a mycobacterium, a fungus, a virus, a parasite or a prion) described herein or known to one skilled in the art. In some embodiments, the infectious disease is caused by bacteria such as, but not limited to, Bacillus anthracis (anthrax), Bordetella pertussis (whooping cough), Chlamydia spp., Corynebacterium diphtheriae (diphtheria), Clostridium tetani (tetanus), Haemophilus influenzae type b, pneumococcus (pneumococcal infections), Streptococcus spp. including Group A or B streptococci, Staphylococci spp., Mycobacterium spp. (e.g., Mycobacterium tuberculosis), Neiserria spp. (e.g., Neiserria meningitidis—meningococcal disease), Salmonella typhi (typhoid), Vibrio cholerae (Cholera), or Yersinia pestis (plague).

In some embodiments, the infectious disease is caused by a virus such as, but not limited to, adenovirus, enterovirus such as polio virus, coronavirus, dengue virus, Ebola virus, herpes viruses such as herpes simplex virus, cytomegalovirus and varicella-zoster (chickenpox and shingles), measles, mumps, rubella, hepatitis-A, -B, or -C, human papilloma virus, Influenza virus, rabies, Japanese encephalitis, rotavirus, human immunodeficiency virus (HIV), respiratory syncytial virus (RSV), smallpox, yellow fever, dengue virus, or Zika Virus. In some embodiments, the infectious disease is caused by a parasite such as, but not limited to, malaria (Plasmodium spp.), Leishmania spp., or a helminth. In some embodiments, the infectious disease is caused by a fungus such as, but not limited to, Candida spp., Aspergillus spp., Cryptococcus spp., Mucormycete spp., Blastomyces dermatitidis, Histoplasma capsulatum, or Sporothrix schenckii. In some embodiments, the infectious disease is caused by a prion. In some embodiments, the infectious disease is sepsis.

In some embodiments, the composition or vaccine composition (e.g., a small molecule formulation or adjuvanted vaccine formulation) may be administered in combination with another therapeutic agent to prevent or treat an infectious disease. Such other therapeutic agents may be, without limitation: antibiotics, anti-viral agents, anti-fungal agents, or anti-parasitic agents. One skilled in the art is familiar with how to select or administer the additional therapeutic agent based on the disease to be treated.

In some embodiments, the disease is an allergy (e.g., allergic rhinitis) or asthma. It has been demonstrated that Th1/Th2 imbalance results in the clinical manifestation of an allergy or asthma (e.g., as described in Ngoc et al., Curr Opin Allergy Clin Immunol. 2005 April; 5(2):161-6, incorporated herein by reference). Administration of the compound of Formula (I) as described herein may be able to restore or partially restore Th1/Th2 balance and possess therapeutic potential for allergies or asthma.

In some embodiments, the disease is cancer. Vaccine compositions comprising cancer-specific antigens and the compound of Formula (I) may be used in cancer immunotherapy by eliciting cancer-specific immune response against the cancer. The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer treated using the composition and methods of the present disclosure is melanoma.

In some embodiments, additional anti-cancer agents may be administered in combination with the composition or vaccine composition described herein. In some embodiments, the anti-cancer agent is selected from the group consisting of: small molecules, oligonucleotides, polypeptides, and combinations thereof. In some embodiments, the anti-cancer agent is a chemotherapeutic agent. In some embodiments, the chemotherapeutic agent is selected from the group consisting of: Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine. In some embodiments, the chemotherapeutic agent is Doxorubicin.

In some embodiments, the anti-cancer agent is an immune checkpoint inhibitor. An “immune checkpoint” is a protein in the immune system that either enhances an immune response signal (co-stimulatory molecules) or reduces an immune response signal. Many cancers protect themselves from the immune system by exploiting the inhibitory immune checkpoint proteins to inhibit the T cell signal. Exemplary inhibitory checkpoint proteins include, without limitation, Cytotoxic T-Lymphocyte-Associated protein 4 (CTLA-4), Programmed Death 1 receptor (PD-1), T-cell Immunoglobulin domain and Mucin domain 3 (TIM3), Lymphocyte Activation Gene-3 (LAG3), V-set domain-containing T-cell activation inhibitor 1 (VTVN1 or B7-H4), Cluster of Differentiation 276 (CD276 or B7-H3), B and T Lymphocyte Attenuator (BTLA), Galectin-9 (GAL9), Checkpoint kinase 1 (Chk1), Adenosine A2A receptor (A2aR), Indoleamine 2,3-dioxygenase (IDO), Killer-cell Immunoglobulin-like Receptor (KIR), Lymphocyte Activation Gene-3 (LAG3), and V-domain Ig suppressor of T cell activation (VISTA).

Some of these immune checkpoint proteins need their cognate binding partners, or ligands, for their immune inhibitory activity. For example, A2AR is the receptor of adenosine A2A and binding of A2A to A2AR activates a negative immune feedback loop. As another example, PD-1 associates with its two ligands, PD-L1 and PD-L2, to down regulate the immune system by preventing the activation of T-cells. PD-1 promotes the programmed cell death of antigen specific T-cells in lymph nodes and simultaneously reduces programmed cell death of suppressor T cells, thus achieving its immune inhibitory function. As yet another example, CTLA4 is present on the surface of T cells, and when bound to its binding partner CD80 or CD86 on the surface of antigen-present cells (APCs), it transmits an inhibitory signal to T cells, thereby reducing the immune response.

An “immune checkpoint inhibitor” is a molecule that prevents or weakens the activity of an immune checkpoint protein, for example, an immune checkpoint inhibitor may inhibit the binding of the immune checkpoint protein to its cognate binding partner, e.g., PD-1, CTLA-4, or A2aR. In some embodiments, the immune checkpoint inhibitor is a small molecule. In some embodiments, the immune checkpoint inhibitors are a nucleic acid aptamer (e.g., a siRNA targeting any one of the immune checkpoint proteins). In some embodiments, the immune checkpoint inhibitor is a recombinant protein. In some embodiments, the immune checkpoint inhibitor is an antibody. In some embodiments, the antibody comprises an anti-CTLA-4, anti-PD-1, anti-PD-L1, anti-TIM3, anti-LAG3, anti-B7-H3, anti-B7-H4, anti-BTLA, anti-GAL9, anti-Chk, anti-A2aR, anti-IDO, anti-KIR, anti-LAG3, anti-VISTA antibody, or a combination of any two or more of the foregoing antibodies. In some embodiments, the immune checkpoint inhibitor is a monoclonal antibody. In some embodiments, the immune checkpoint inhibitor comprises anti-PD1, anti-PD-L1, anti-CTLA-4, or a combination of any two or more of the foregoing antibodies. For example, the anti-PD-1 antibody is pembrolizumab (Keytruda®) or nivolumab (Opdivo®) and the anti-CTLA-4 antibody is ipilimumab (Yervoy®). Thus, in some embodiments, the immune checkpoint inhibitor comprises pembrolizumab, nivolumab, ipilimumab, or any combination of two or more of the foregoing antibodies. The examples described herein are not meant to be limiting and that any immune checkpoint inhibitors known in the art and any combinations thereof may be used in accordance with the present disclosure.

Additional exemplary agents that may be used in combination with the compositions described herein include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In some embodiments, the additional agent is an anti-proliferative agent (e.g., anti-cancer agent). In some embodiments, the additional pharmaceutical agent is an anti-leukemia agent. In some embodiments, the additional pharmaceutical agent is selected from ABITREXATE (methotrexate), ADE, Adriamycin RDF (doxorubicin hydrochloride), Ambochlorin (chlorambucil), ARRANON (nelarabine), ARZERRA (ofatumumab), BOSULIF (bosutinib), BUSULFEX (busulfan), CAMPATH (alemtuzumab), CERUBIDINE (daunorubicin hydrochloride), CLAFEN (cyclophosphamide), CLOFAREX (clofarabine), CLOLAR (clofarabine), CVP, CYTOSAR-U (cytarabine), CYTOXAN (cyclophosphamide), ERWINAZE (Asparaginase Erwinia chrysanthemi), FLUDARA (fludarabine phosphate), FOLEX (methotrexate), FOLEX PFS (methotrexate), GAZYVA (obinutuzumab), GLEEVEC (imatinib mesylate), Hyper-CVAD, ICLUSIG (ponatinib hydrochloride), IMBRUVICA (ibrutinib), LEUKERAN (chlorambucil), LINFOLIZIN (chlorambucil), MARQIBO (vincristine sulfate liposome), METHOTREXATE LPF (methorexate), MEXATE (methotrexate), MEXATE-AQ (methotrexate), mitoxantrone hydrochloride, MUSTARGEN (mechlorethamine hydrochloride), MYLERAN (busulfan), NEOSAR (cyclophosphamide), ONCASPAR (Pegaspargase), PURINETHOL (mercaptopurine), PURIXAN (mercaptopurine), Rubidomycin (daunorubicin hydrochloride), SPRYCEL (dasatinib), SYNRIBO (omacetaxine mepesuccinate), TARABINE PFS (cytarabine), TASIGNA (nilotinib), TREANDA (bendamustine hydrochloride), TRISENOX (arsenic trioxide), VINCASAR PFS (vincristine sulfate), ZYDELIG (idelalisib), or a combination thereof. In some embodiments, the additional pharmaceutical agent is an anti-lymphoma agent. In some embodiments, the additional pharmaceutical agent is ABITREXATE (methotrexate), ABVD, ABVE, ABVE-PC, ADCETRIS (brentuximab vedotin), ADRIAMYCIN PFS (doxorubicin hydrochloride), ADRIAMYCIN RDF (doxorubicin hydrochloride), AMBOCHLORIN (chlorambucil), AMBOCLORIN (chlorambucil), ARRANON (nelarabine), BEACOPP, BECENUM (carmustine), BELEODAQ (belinostat), BEXXAR (tositumomab and iodine I 131 tositumomab), BICNU (carmustine), BLENOXANE (bleomycin), CARMUBRIS (carmustine), CHOP, CLAFEN (cyclophosphamide), COPP, COPP-ABV, CVP, CYTOXAN (cyclophosphamide), DEPOCYT (liposomal cytarabine), DTIC-DOME (dacarbazine), EPOCH, FOLEX (methotrexate), FOLEX PFS (methotrexate), FOLOTYN (pralatrexate), HYPER-CVAD, ICE, IMBRUVICA (ibrutinib), INTRON A (recombinant interferon alfa-2b), ISTODAX (romidepsin), LEUKERAN (chlorambucil), LINFOLIZIN (chlorambucil), Lomustine, MATULANE (procarbazine hydrochloride), METHOTREXATE LPF (methotrexate), MEXATE (methotrexate), MEXATE-AQ (methotrexate), MOPP, MOZOBIL (plerixafor), MUSTARGEN (mechlorethamine hydrochloride), NEOSAR (cyclophosphamide), OEPA, ONTAK (denileukin diftitox), OPPA, R-CHOP, REVLIMID (lenalidomide), RITUXAN (rituximab), STANFORD V, TREANDA (bendamustine hydrochloride), VAMP, VELBAN (vinblastine sulfate), VELCADE (bortezomib), VELSAR (vinblastine sulfate), VINCASAR PFS (vincristine sulfate), ZEVALIN (ibritumomab tiuxetan), ZOLINZA (vorinostat), ZYDELIG (idelalisib), or a combination thereof. In some embodiments, the additional pharmaceutical agent is REVLIMID (lenalidomide), DACOGEN (decitabine), VIDAZA (azacitidine), CYTOSAR-U (cytarabine), IDAMYCIN (idarubicin), CERUBIDINE (daunorubicin), LEUKERAN (chlorambucil), NEOSAR (cyclophosphamide), FLUDARA (fludarabine), LEUSTATIN (cladribine), or a combination thereof. In some embodiments, the additional pharmaceutical agent is ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), ST1VARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC220, dovitinib lactate (TKI258, CHIR-258), BIBW 2992 (TOVOKTM), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In some embodiments, the additional agent is a protein kinase inhibitor (e.g., tyrosine protein kinase inhibitor). In some embodiments, the additional agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation.

In some embodiments, a composition or vaccine composition described herein is formulated for administration to a subject. In some embodiments, the composition or vaccine composition further comprises a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the composition or vaccine composition described herein also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The composition or vaccine composition described herein may conveniently be presented in unit dosage form and may be prepared by any of the methods well-known in the art of pharmacy. The term “unit dose” when used in reference to a composition or vaccine composition described herein of the present disclosure refers to physically discrete units suitable as unitary dosage for the subject, each unit containing a predetermined quantity of active material calculated to produce the desired therapeutic effect in association with the required diluent; i.e., carrier, or vehicle. Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij© 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum©), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof. The formulation of the composition or vaccine composition described herein may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragées, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical Mantoux method of intradermal administration.

Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

For topical administration, the composition or vaccine composition described herein can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to deliver therapeutic levels of the active ingredient for at least 30 days, and preferably 60 days. Long-term sustained release implants are well-known to those of ordinary skill in the art and include some of the release systems described above.

In some embodiments, the composition or vaccine composition described herein used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The cyclic Psap peptide and/or the composition or vaccine composition described herein ordinarily will be stored in lyophilized form or as an aqueous solution if it is highly stable to thermal and oxidative denaturation. The pH of the preparations typically will be about from 6 to 8, although higher or lower pH values can also be appropriate in certain instances. The chimeric constructs of the present disclosure can be used as vaccines by conjugating to soluble immunogenic carrier molecules. Suitable carrier molecules include protein, including keyhole limpet hemocyanin, which is a preferred carrier protein. The chimeric construct can be conjugated to the carrier molecule using standard methods. (Hancock et al., “Synthesis of Peptides for Use as Immunogens,” in Methods in Molecular Biology: Immunochemical Protocols, Manson (ed.), pages 23-32 (Humana Press 1992)).

In some embodiments, the present disclosure contemplates a vaccine composition comprising a pharmaceutically acceptable injectable vehicle. The vaccines of the present disclosure may be administered in conventional vehicles with or without other standard carriers, in the form of injectable solutions or suspensions. The added carriers might be selected from agents that elevate total immune response in the course of the immunization procedure.

Liposomes have been suggested as suitable carriers. The insoluble salts of aluminum, that is aluminum phosphate or aluminum hydroxide, have been utilized as carriers in routine clinical applications in humans. Polynucleotides and polyelectrolytes and water-soluble carriers such as muramyl dipeptides have been used.

Preparation of injectable vaccines of the present disclosure, includes mixing the antigen and/or the compound of Formula (I) with muramyl dipeptides or other carriers. The resultant mixture may be emulsified in a mannide monooleate/squalene or squalane vehicle. Four parts by volume of squalene and/or squalane are used per part by volume of mannide monooleate. Methods of formulating vaccine compositions are well known to those of ordinary skill in the art. (Rola, Immunizing Agents and Diagnostic Skin Antigens. In: Remington's Pharmaceutical Sciences, 18th Edition, Gennaro (ed.), (Mack Publishing Company 1990) pages 1389-1404).

Additional pharmaceutical carriers may be employed to control the duration of action of a vaccine in a therapeutic application. Control release preparations can be prepared through the use of polymers to complex or adsorb chimeric construct. For example, biocompatible polymers include matrices of poly(ethylene-co-vinyl acetate) and matrices of a polyanhydride copolymer of a stearic acid dimer and sebacic acid. (Sherwood et al. (1992) Bio/Technology 10: 1446). The rate of release of the chimeric construct from such a matrix depends upon the molecular weight of the construct, the amount of the construct within the matrix, and the size of dispersed particles. (Saltzman et al. (1989) Biophys. J. 55: 163; Sherwood et al, supra.; Ansel et al. Pharmaceutical Dosage Forms and Drug Delivery Systems, 5th Edition (Lea & Febiger 1990); and Gennaro (ed.), Remington's Pharmaceutical Sciences, 18th Edition (Mack Publishing Company 1990)). The chimeric construct can also be conjugated to polyethylene glycol (PEG) to improve stability and extend bioavailability times (e.g., Katre et al.; U.S. Pat. No. 4,766,106).

### Kits

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, inflammatory disease, autoimmune disease, infectious disease, or chronic disease) in a subject in need thereof. In certain embodiments, the kits are useful for preventing a disease (e.g., proliferative disease, inflammatory disease, autoimmune disease, infectious disease, or chronic disease) in a subject in need thereof. In certain embodiments, the kits are useful as enhancers of an immune response (e.g., innate and/or adaptive immune response), and/or adjuvants in a vaccine for a disease, (e.g., proliferative disease, inflammatory disease, autoimmune disease, infectious disease, or chronic disease) in a subject, biological sample, tissue, or cell.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, inflammatory disease, autoimmune disease, infectious disease, or chronic disease) in a subject in need thereof. In certain embodiments, the kits and instructions provide for preventing a disease (e.g., proliferative disease, inflammatory disease, autoimmune disease, infectious disease, or chronic disease) in a subject in need thereof. In certain embodiments, the kits and instructions provide for enhancing of an immune response (e.g., innate and/or adaptive immune response) in a subject, biological sample, tissue, or cell. In certain embodiments, the kits and instructions provide for use of the compounds as adjuvants in a vaccine for a disease, (e.g., proliferative disease, inflammatory disease, autoimmune disease, infectious disease, or chronic disease) in a subject, biological sample, tissue, or cell. A kit described herein may include one or more additional pharmaceutical agents described herein as a separate composition.

### Administration of Immunostimulatory Compositions

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. Prophylactic treatment refers to the treatment of a subject who is not and was not with a disease but is at risk of developing the disease or who was with a disease, is not with the disease, but is at risk of regression of the disease. In some embodiments, the subject is at a higher risk of developing the disease or at a higher risk of regression of the disease than an average healthy member of a population.

An “effective amount” of a composition described herein refers to an amount sufficient to elicit the desired biological response. An effective amount of a composition described herein may vary depending on such factors as the desired biological endpoint, the pharmacokinetics of the compound, the condition being treated, the mode of administration, and the age and health of the subject. In some embodiments, an effective amount is a therapeutically effective amount. In some embodiments, an effective amount is a prophylactic treatment. In some embodiments, an effective amount is the amount of a compound described herein in a single dose. In some embodiments, an effective amount is the combined amounts of a compound described herein in multiple doses. When an effective amount of a composition is referred herein, it means the amount is prophylactically and/or therapeutically effective, depending on the subject and/or the disease to be treated. Determining the effective amount or dosage is within the abilities of one skilled in the art.

The terms “administer,” “administering,” or “administration” refers to implanting, absorbing, ingesting, injecting, inhaling, or otherwise introducing a compound described herein, or a composition thereof, in or on a subject. The composition of the vaccine composition described herein may be administered systemically (e.g., via intravenous injection) or locally (e.g., via local injection). In some embodiments, the composition of the vaccine composition described herein is administered orally, intravenously, topically, intranasally, or sublingually.

Parenteral administrating is also contemplated. The term “parenteral” as used herein includes subcutaneous, intracutaneous, intravenous, intramuscular, intraarticular, intraarterial, intrasynovial, intrasternal, intrathecal, intralesional, intradermally, and intracranial injection or infusion techniques. In some embodiments, the administering is done intramuscularly, intradermally, orally, intravenously, topically, intranasally, intravaginally, or sublingually. In some embodiments, the composition is administered prophylactically.

In some embodiments, the composition or vaccine composition is administered once or administered repeatedly (e.g., 2, 3, 4, 5, or more times). For multiple administrations, the administrations may be done over a period of time (e.g., 6 months, a year, 2 years, 5 years, 10 years, or longer). In some embodiments, the composition or vaccine composition is administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later).

A “subject” to which administration is contemplated refers to a human (i.e., male or female of any age group, e.g., pediatric subject (e.g., infant, child, or adolescent) or adult subject (e.g., young adult, middle-aged adult, or senior adult) or non-human animal. In some embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In some embodiments, the non-human animal is a fish, reptile, or amphibian. The non-human animal may be a male or female at any stage of development. The non-human animal may be a transgenic animal or genetically engineered animal.

A “subject in need thereof” refers to a human subject in need of treatment of a disease or in need of reducing the risk of developing a disease. In some embodiments, the subject has any of the diseases described herein (e.g., infectious disease, cancer, or allergy). In some embodiments, the subject is at risk of developing any of the diseases described herein (e.g., infectious disease, cancer, or allergy). In some embodiments, administering the compound of Formula (I) or the compound of Formula (I) and an antigen as described herein to a subject having a disease treats the disease (therapeutic use). In some embodiments, administering the compound of Formula (I) or the compound of Formula (I) and an antigen as described herein to a subject at risk of developing a disease reduces the likelihood (e.g., by 20%, 30%, 40%, 50%, 60%, 70%, 80%, 90%, 95%, 99% or more) of the subject developing the disease (prophylactic use). In some embodiments, a subject has or is at risk for an infectious disease caused by a pathogen (e.g., an infectious bacterium, a virus, a parasite, or a fungus). In some embodiments, a subject has or is at risk for an infectious disease caused by a Beta Coronavirus, such as MERS-CoV, SARS-CoV-1, or SARS-CoV-2.

In some embodiments, the present disclosure contemplates the vaccination of human infants or neonates. In some embodiments, the subject is a human infant. In some embodiments, the human infant is a neonate that is less than 28 days of age. In some embodiments, the human infant is less than 1, 1, 2, 3, 4, 5, 6, 7, 8, 9, 10, 11, 12, 13, 14, 15, 16, 17, 18, 19, 20, 21, 22, 23, 24, 25, 26, 27, or 28 days of age at the time of administration of the vaccine composition described herein (i.e., vaccination).

In some embodiments, the human subject is more than 28 days of age (e.g., 1 month, 2 months, 3 months, 4 months, 5 months, 6 months, 7 months, 8 months, 9 months, 10 months, 11 months, 12 months, 2 years, 3 years, 4 years, 5 years, 10 years, 11 years, 12 years, 13 years, 14 years, 15 years, 16 years, 17 years of age). In some embodiments, the human subject is an adult (e.g., more than 18 years of age). In some embodiments, the human subject is an elderly subject (e.g., more than 60 years of age). In some embodiments, the human subject is 60 years, 65 years, 70 years, 75 years, 80 years, 85 years, 90 years, 95 years, 100 years, or more than 100 years of age.

In some embodiments, a human subject receives 1, 2, or more than 2 doses of the vaccine described herein. In some embodiments, a human neonate receives one dose before 28 days of age (e.g., less than 1, 1, 2, 3, 4, 5, 6, 7, 8, 9, 10, 11, 12, 13, 14, 15, 16, 17, 18, 19, 20, 21, 22, 23, 24, 25, 26, 27 days of age) and a second dose before, at, or after 28-days of age. In some embodiments, a human subject receives one dose before 60 years of age and a second dose before, at, or after 60 years of age (e.g., 60, 65, 70, 75, 80, 85, 90, 95, 100, or more than 100 years of age, or any age therebetween as if explicitly recited). In some embodiments, the human subject receives a second dose of the vaccine 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or 10 years or more after receiving the first dose.

In some embodiments, the human subject is part of one or more immunologically vulnerable populations. In some embodiments, the human subject is frail (e.g., a subject having frailty syndrome, a malnourished subject, or a subject with a chronic disease causing frailty). In some embodiments, the human subject has a weak immune system, such as an undeveloped (e.g., an infant or a neonate subject), immunosenescent (e.g., an elderly subject), or compromised immune system. Immunosenescent subjects include, without limitation, subjects exhibiting a decline in immune function associated with advanced age. Immunocompromised subjects include, without limitation, subjects with primary immunodeficiency or acquired immunodeficiency such as those suffering from sepsis, HIV infection, and cancers, including those undergoing chemotherapy and/or radiotherapy, as well as subjects to which immunosuppressants are administered, as for organ or tissue transplantation. In some embodiments, the human subject has or is suspected of having one or more disorders or diseases that reduce immune system function and/or increase the risk of infection in the subject by one or more pathogens (e.g., a bacterium, a mycobacterium, a fungus, a virus, a parasite, or a prion). In some embodiments, the human subject is, for example, a subject that has or is suspected of having chronic lung disease, asthma, cardiovascular disease, cancer, a metabolic disorder (e.g., obesity or diabetes mellitus), chronic kidney disease, or liver disease.

In some embodiments, the subject is a companion animal (a pet). The use of the compound of Formula (I) described herein in a veterinary vaccine is also within the scope of the present disclosure. “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include: rodents (e.g., rats, mice, guinea pigs, and hamsters), rabbits, or non-human primates.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1: Identification of PVP-179, a Small Molecule Adjuvant for Eliciting an Innate Immune Response

A small molecule adjuvant is reported that is able to induce Th1-polarizing cytokine production by human adult and elder mononuclear cells in vitro (e.g., TNF) and enhance antibody responses to SARS-CoV-2 spike protein in elder mice in vivo. This pharmacophore has demonstrated activity across age groups, particularly with strong responses in elderly human leukocytes in vitro and effective adjuvantation in elder mice, with a putative mechanism of action via activation of Toll-like receptor (TLR)-8. Adjuvants are able to enhance, prolong, and modulate immune responses to vaccinal antigens to maximize protective immunity, provide dose sparing effects, and may potentially enable effective immunization in vulnerable populations. In this context, a targeted screen was undertaken to identify small molecules capable of modifying elderly human immune responses.

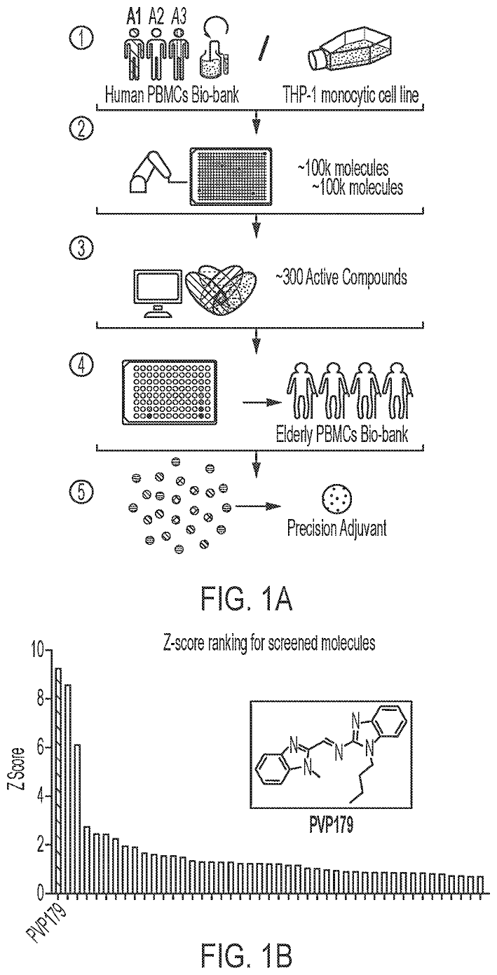

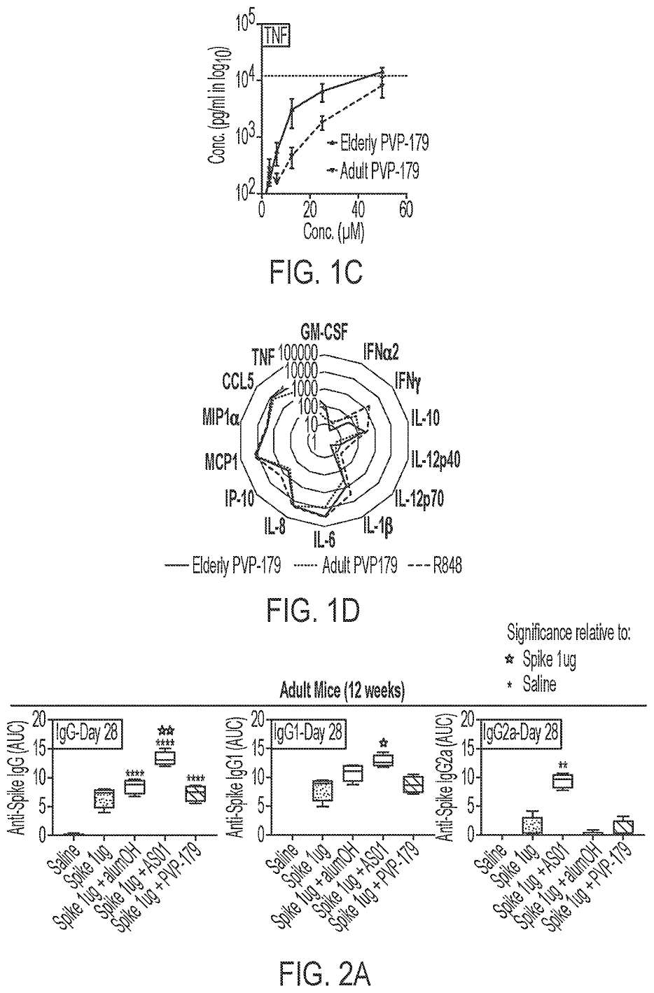

In a previous high throughput screen (HTS), a number of hit molecules were identified as potentially activating human leukocytes. Of 200,000 molecules screened in a human monocytic THP-1 cell line, 177 demonstrated NF-kB/IRF-inducing and/or leukocyte adherence-inducing activity. A separate pilot screen of 9,000 of the molecules for TNF-inducing activity in adult human peripheral blood mononuclear cells (PBMCs) subsequently identified 133 molecules. These molecules originated from various bioactive and commercial libraries such as ChemDiv, ChemBridge, and Asinex. All libraries were owned and provided by the Institute of Chemistry and Cell Biology (ICCB)—Longwood (Harvard Medical School). The screening approach was to assemble all 310 molecules onto a 384-well chemical plate and assess whether these molecules could stimulate TNF production by human elderly PBMCs (FIG. 1A). From this screen, a small molecule with robust activity toward elderly PBMCs was confirmed by TNF measurements. This molecule was referred to as Precision Vaccines Program molecule #179 (i.e., the 179th molecule ordered into the Program), or “PVP-179”, which was both a positive hit in the original THP-1 screen and a robust TNF-inducing hit in the subsequent screen with elderly PBMCs (FIG. 1B). According to PubChem (https://pubchem.ncbi.nlm.nih.gov/compound/2849896), the SMILES for PVP-179 is: “CCCCN1C2=CC═CC═C2N═CN═C═CC═CC═C4N3C”.

The activity of PVP-179 was confirmed in a 96-well format in both human adult and elderly PBMCs. PVP-179 induces concentration-dependent production of TNF in both elderly and adult PBMCs when stimulated for 24 hours (FIG. 1C). Cytokine responses were investigated by a 14-multiplex assay, which demonstrated that PVP-179 tends to induce a greater innate immune response in elder leukocytes than in younger adult controls. PVP-179 induced titration-dependent concentration of proinflammatory cytokines (e.g., TNF, IL-6, IL-8, IL-10, IL-12 p40, IL-12 p70, IP-10, CCL5, CCL3, MCP1, GM-CSF, IFN-γ, IFN-αt2, and IL1-β) similarly to R848, a commercially available TLR7/8 agonist, administered at a concentration of 25 μM, demonstrating broad cytokine and chemokine inducing activity (FIG. 1D).

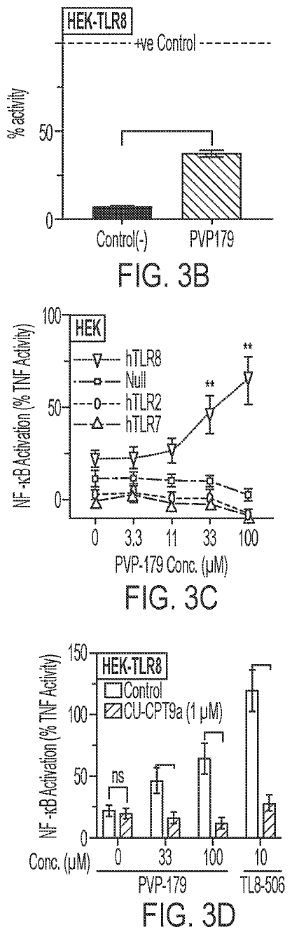

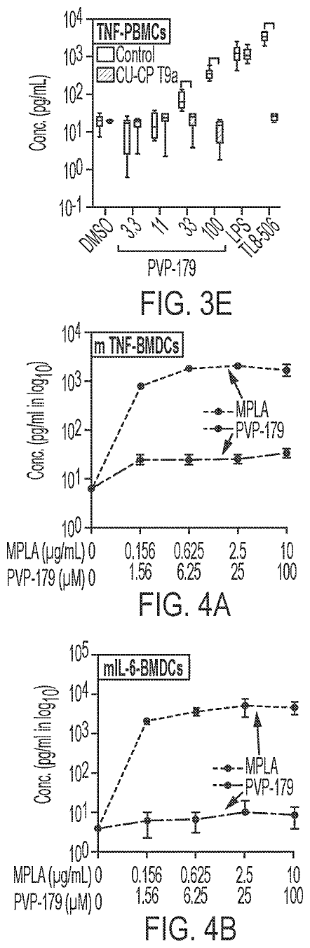

Subsequent pattern recognition receptor (PRR) ligand screening using HEK cell reporter cells indicated that PVP-179 has maximal activity toward human (h)TLR8, among all TLRs and other receptor tested (FIGS. 3A-3B). PVP-179 demonstrated concentration-dependent activity in HEK cells expressing hTLR8, however, no activity was detected in HEK cells lines expressing hTLR2 or hTLR7, or in the null HEK cells (negative control cells carrying only the reporter gene), even when tested at higher concentrations (FIG. 3C). Next a TLR8-specific pharmacological inhibitor (CUCPT-9a), which stabilizes the TLR8 dimer in its resting state, was used to inhibit activity downstream of TLR8. In HEK-hTLR8 cell lines, activity of PVP-179 was significantly blocked at concentrations of 33 μM or above (FIG. 3D). Similarly, CUCPT-9a completely blocked PVP-179 induced TNF production in human adult PBMCs, suggesting TLR8 dependence of PVP-179 to induce cytokine response (FIG. 3E).

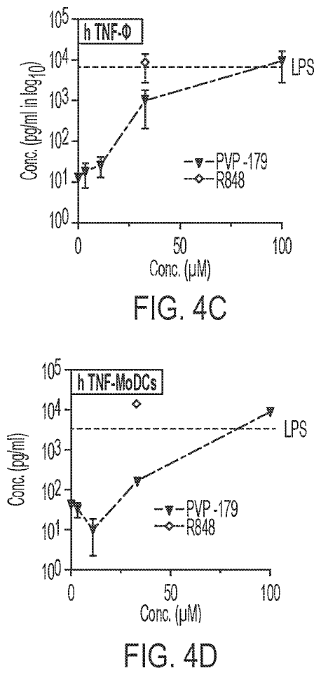

The activity profile of PVP-179 was assessed in multiple primary cell types and a distinct profile was observed, suggesting human TLR8 dependence. While PVP-179 demonstrated minimal cytokine inducing activity towards murine bone marrow-derived dendritic cells (FIGS. 4A-4B), it demonstrated concentration-dependent activity towards human monocyte derived macrophages (FIG. 4C) and dendritic cells (FIG. 4D).

### Example 2: Adjuvantation of SARS-CoV-2 Spike Protein with PVP-179

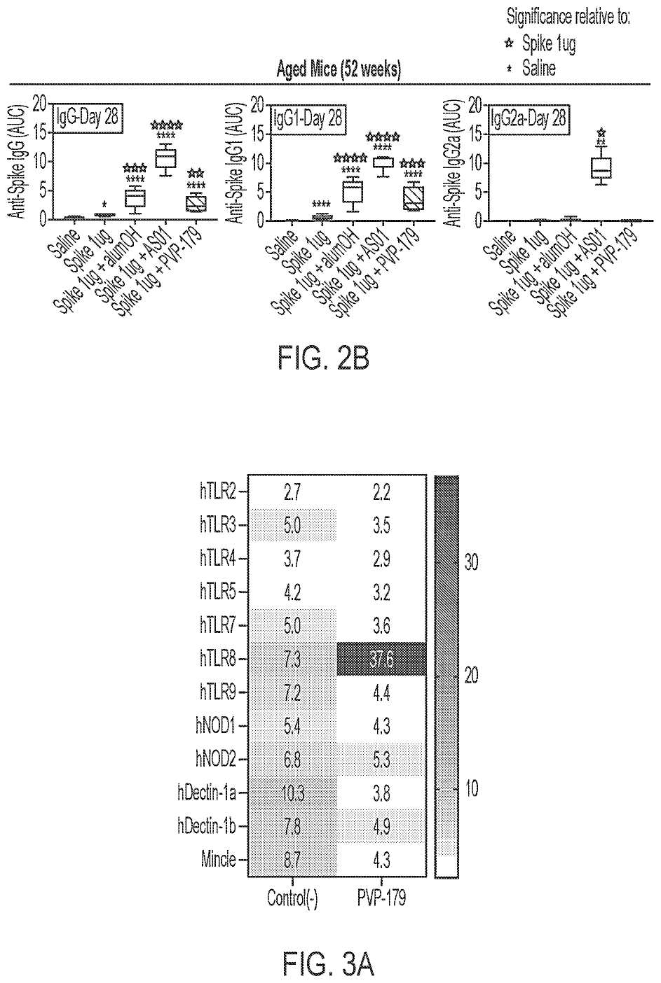

The adjuvant activity of PVP-179 was further evaluated in a Balb/c murine model of SARS-CoV-2 immunization. Mice were immunized with recombinant SARS-CoV-2 spike protein (rSpike) either with or without PVP-179 and received a booster dose on Day 14 post-immunization. Immunization of rSpike with PVP-179 was further compared with immunization of rSpike adjuvanted with aluminum hydroxide (alumOH) or ASO1, a liposomal adjuvant combining 3-O-desacyl-4′-monophosphoryl lipid A (MPL) and a saponin, QS-21, which is known to activate TLR4. The addition of PVP-179 enhanced rSpike immunogenicity in both adult (12 weeks) and aged mice (52 weeks/12 months old). Aged mice vaccinated with PVP-179-adjuvanted rSpike demonstrated significantly higher antibody titers (˜2-5-fold) compared to elderly mice vaccinated with rSpike alone (FIG. 2B). PVP-179 also demonstrated adjuvant activity in adult mice (FIG. 2A). In aged mice, adjuvantation with PVP-179 primarily enhanced the production of anti-rSpike IgG1 subtype antibodies.

These results described above provide convincing proof-of-concept that PVP-179 is an effective adjuvant for augmenting immune responses to administered antigens, such as those of pathogens, especially in elderly subjects. This is significant due to the unmet need for vaccines that are highly effective in elderly humans. Potentially, vaccines, such as those for SARS-CoV-2, could be adjuvanted with PVP-179 and used to provide stronger and/or longer lasting protection than is otherwise currently available to this immunologically vulnerable population. Improved adjuvantation may also enable these vaccines to be administered as a single dose, rather than in two or more doses, allowing for more efficient vaccination programs. It should also be noted that as TLR8 is endogenously stimulated by single stranded RNA (ssRNA), such as that comprised by particular viruses, the development of small molecular TLR8 agonists like PVP-179 may have significant advantages compared to other TLR8 agonists. For example, PVP-179 may be more efficacious in certain populations, such as the elderly, neonatal, or immunocompromised, and may be more amenable to mass production. In addition, a small molecule such as PVP-179 that is able to activate robust elderly immune responses has substantial potential in the field of immune-oncology, potentially overcoming immune senescence to enable an effective immune response to clear cancerous cells.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of enhancing an immune response in a subject in need thereof, the method comprising administering to the subject an effective amount of a compound of Formula (I):

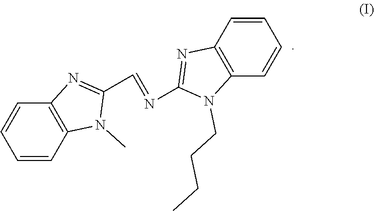 [SMILES: CCCCn1c(/N=C/c2nc3ccccc3n2C)nc2ccccc21]

2. A method of treating a disease or reducing the risk of a disease, the method comprising administering to a subject in need thereof an effective amount of a compound of Formula (I):

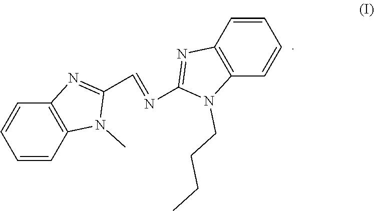 [SMILES: CCCCn1c(/N=C/c2nc3ccccc3n2C)nc2ccccc21]

3. The method of claim 1, wherein the immune response is an innate immune response.

4. The method of claim 1, wherein the immune response is an adaptive immune response.

5. The method of any one of claims 1, 3, or 4, wherein the immune response is a cell-mediated immune response.

6. The method of any one of claims 1, 3, 4, or 5, wherein the immune response is a heterologous immune response.

7. The method of any one of claims 1-4, wherein the compound is adsorbed onto alum.

8. The method of any one of claims 1-7, wherein the compound is lipidated.

9. The method of any one of claims 1-8, wherein the compound is in an aqueous formulation.

10. The method of any one of claims 1-8, wherein the compound is in an emulsion formulation and/or a nanoparticle formulation.

11. The method of any one of claims 1-9, wherein the compound is administered to the subject more than once.

12. The method of any one of claims 1-11, wherein the subject has or is at risk of developing an infectious disease.

13. The method of claim 12, wherein the infectious disease is caused by a bacterium, a mycobacterium, a fungus, a virus, a parasite, or a prion.

14. The method of claim 11 or claim 13, wherein the infectious disease is sepsis.

15. The method of any one of claims 1-11, wherein the subject has or is at risk of developing cancer.

16. The method of claim 15, wherein the cancer is metastatic cancer.

17. The method of claim 15 or claim 16, wherein the cancer is a hematological cancer, lung cancer, breast cancer, brain cancer, gastrointestinal cancer, liver cancer, kidney cancer, bladder cancer, pancreatic cancer, ovarian cancer, testicular cancer, prostate cancer, endometrial cancer, muscle cancer, bone cancer, neuroendocrine cancer, connective tissue cancer, head and neck cancer, or skin cancer.

18. The method of any one of claims 1-11, wherein the subject has or is at risk of developing an allergy.

19. The method of any one of claims 1-11, wherein the subject has a radiation injury.

20. The method of any one of claims 1-19, wherein the subject is immune-senescent, immune-compromised, is infected with human immunodeficiency virus (HIV), has chronic lung disease, asthma, cardiovascular disease, cancer, a metabolic disorder, chronic kidney disease, liver disease, is malnourished, or is frail.

21. The method of any one of claims 1-20, wherein the administration is systemic or local.

22. The method of any one of claims 1-21, wherein the administration is intramuscular, intradermal, oral, intravenous, topical, intranasal, intravaginal, or sublingual.

23. The method of any one of claims 1-22, wherein the administration is prophylactic.

24. The method of any one of claims 1-23, wherein the subject is a human neonate, an infant, an adult, or an elderly individual.

25. The method of claim 24, wherein the subject is a human adult.

26. The method of claim 24, wherein the subject is an elderly human.

27. The method of claim 26, wherein the administration occurs when the subject is more than 65 years of age.

28. The method of any one of claims 1-23, wherein the subject is a companion animal or a research animal.

29. The method of claim 28, wherein the subject is an adult or elderly companion animal.

30. The method of any one of claims 1-29, wherein the compound activates peripheral blood mononuclear cells (PBMCs).

31. The method of claim 30, wherein the PBMCs are macrophages or dendritic cells.

32. The method of any one of claims 1-31, wherein the compound induces a T helper type 1 (Th1) immune response.

33. The method of any one of claims 1-32, wherein the compound induces the production of a signaling molecule selected from a proinflammatory or Th-polarizing cytokine or chemokine in the subject.

34. The method of claim 33, wherein the signaling molecule is a cytokine selected from TNF, IL-6, IL-10, IL-12 p40, IL-12 p70, GM-CSF, IFN-7, IFN-α2, IL1-β and/or IP-10, a chemokine selected from CCL2 (MCP1), CCL3, CCL5, and/or CXCL8 (IL-8), or a combination thereof.

35. The method of any one of claims 1-34, wherein the compound of enhances humoral immunity.

36. The method of any one of claims 1-35, wherein the compound induces the production of an immunoglobulin in the subject.

37. The method of claim 36, wherein the immunoglobulin is an immunoglobulin G (IgG), immunoglobulin A (IgA), or immunoglobulin M (IgM).

38. The method of claim 37, wherein the IgG is a subclass 1 IgG (IgG1) or a subclass 2 IgG (IgG2).

39. A composition comprising an antigen and a compound of Formula (I):

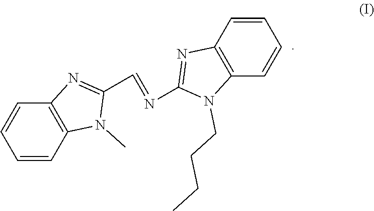 [SMILES: CCCCn1c(/N=C/c2nc3ccccc3n2C)nc2ccccc21]

40. The composition of claim 39, wherein the antigen comprises a protein or polypeptide.

41. The composition of claim 39 or claim 40, wherein the antigen comprises a nucleic acid encoding a protein or a polypeptide.

42. The composition of claim 41, wherein the nucleic acid is DNA or RNA.

43. The composition of any one of claims 39-42, wherein the antigen is from a microbial pathogen.

44. The composition of claim 43, wherein the microbial pathogen is a bacterium, mycobacterium, fungus, virus, parasite, or prion.

45. The composition of claim 44, wherein the bacterium is Bacillus anthracis, Bordetella pertussis, Corynebacterium diphtheriae, Clostridium tetani, Haemophilus influenzae type b, pneumococcus, Staphylococci spp., Streptococcus spp., Mycobacterium spp., Neiserria spp., Salmonella typhi, Vibrio cholerae, or Yersinia pestis.

46. The composition of claim 44, wherein the virus is an adenovirus, a coronavirus, an enterovirus such as polio virus, dengue virus, Ebola virus, a herpes viruses such as herpes simplex virus, cytomegalovirus and varicella-zoster, measles, mumps, rubella, hepatitis A virus, hepatitis B virus, hepatitis C virus, human papilloma virus, Influenza virus, rabies, Japanese encephalitis, rotavirus, human immunodeficiency virus (HIV), respiratory syncytial virus (RSV), smallpox, yellow fever, dengue virus, or Zika virus.

47. The composition of claim 46, wherein the coronavirus virus is Middle East Respiratory Syndrome coronavirus (MERS-CoV), Severe Acute Respiratory Syndrome (SARS)-associated coronavirus (SARS-CoV)-1, or SARS-CoV-2.

48. The composition of claim 47, wherein the antigen is a MERS-CoV spike protein, a SARS-CoV-1 spike protein, or a SARS-CoV-2 spike protein.

49. The composition of claim 47, wherein the antigen is a MERS-CoV spike protein receptor binding domain (RBD), a SARS-CoV-1 spike protein RBD, or a SARS-CoV-2 spike protein RBD.

50. The composition of claim 47, wherein the antigen is a nucleic acid encoding a MERS-CoV spike protein, a MERS-CoV spike protein RBD, a SARS-CoV-1 spike protein, a SARS-CoV-1 spike protein RBD, a SARS-CoV-2 spike protein, or a SARS-CoV-2 spike protein RBD.

51. The composition of claim 47, wherein the antigen comprises a viral particle of MERS-CoV, SARS-CoV-1, or SARS-CoV-2.

52. The composition of claim 47, wherein the antigen comprises killed or inactivated MERS-CoV, SARS-CoV-1, or SARS-CoV-2.

53. The composition of claim 47, wherein the antigen comprises live attenuated MERS-CoV, SARS-CoV-1, or SARS-CoV-2.

54. The composition of claim 44, wherein the parasite is Plasmodium spp., Leishmania, or a helminth.

55. The composition of claim 44, wherein the fungus is Candida spp., Aspergillus spp., Cryptococcus spp., Mucormycete, Blastomyces dermatitidis, Histoplasma capsulatum, or Sporothrix schenckii.

56. The composition of any one of claims 39-42, wherein the antigen is a cancer-specific antigen.

57. The composition of claim 56, wherein the antigen is a heteroclitic epitope or a cryptic epitope derived from the cancer-specific antigen.

58. The composition of claim 56, wherein the cancer-specific antigen is a neoantigen.

59. The composition of any one of claims 39-58, wherein the antigen comprises a lipopolysaccharide (LPS).

60. The composition of any one of claims 39-59, wherein the compound is conjugated to the antigen.

61. The composition of any one of claims 39-59, wherein the compound is not conjugated to the antigen.

62. The composition of any one of claims 39-61, further comprising a pharmaceutically acceptable carrier.

63. The composition of any one of claims 39-55, wherein the composition is a vaccine composition.

64. The composition of claim 63, wherein the compound is an adjuvant.

65. The composition of claim 63 or claim 64, wherein the antigen is adsorbed onto alum.

66. The composition of any one of claims 63-65, wherein the compound is adsorbed onto alum.

67. The composition of any one of claims 63-66, wherein the vaccine composition further comprises a second adjuvant.

68. The composition of claim 67, wherein second adjuvant is an agonist of a Pattern Recognition Receptor (PRR).

69. The composition of claim 68, wherein the PRR is selected from the group consisting of Toll-like receptors (TLRs), NOD-like receptors (NLRs), RIG-I-like receptor (RLR), C-type Lectin receptors (CLRs), and a stimulator of interferon genes (STING).

70. The composition of any one of claims 67-69, wherein second adjuvant is bound to or adsorbed to alum.

71. The composition of claim 67, wherein the second adjuvant is alum.

72. The composition of any one of claims 67-71, wherein the second adjuvant is an emulsion.

73. The composition of any one of claims 63-72, wherein the vaccine composition is a subunit vaccine, an attenuated vaccine, or a conjugate vaccine.

74. A method of enhancing an immune response to an antigen in a subject in need thereof, the method comprising administering to the subject an effective amount of the composition of any one of claims 39-73.

75. The method of claim 74, wherein the subject is a human neonate, an infant, an adult, or an elderly individual.

76. The method of claim 75, wherein the subject is a human adult.

77. The method of claim 75, wherein the subject is an elderly human.

78. The method of claim 77, wherein the administration occurs when the subject is more than 65 years of age.

79. The method of any one of claims 74-78, wherein the subject is immune-senescent, immune-compromised, is infected with human immunodeficiency virus (HIV), has chronic lung disease, asthma, cardiovascular disease, cancer, a metabolic disorder, chronic kidney disease, liver disease, is malnourished, or is frail.

80. The method of any one of claims 74-79, wherein the subject is infected with SARS-CoV-2.

81. The method of any one of claims 74-79, wherein the subject is at risk for SARS-CoV-2.

82. The method of any one of claims 74-81, wherein the composition induces a cell-mediated immune response.

83. The method of any one of claims 74-82, wherein the composition induces a heterologous immune response.

84. The method of any one of claims 74-83, wherein the composition induces a T helper type 1 (Th1) immune response.

85. The method of any one of claims 74-84, wherein the composition induces the production of a signaling molecule selected from a proinflammatory or Th-polarizing cytokine or chemokine in the subject.

86. The method of claim 85, wherein the signaling molecule is a cytokine selected from TNF, IL-6, IL-10, IL-12 p40, IL-12 p70, GM-CSF, IFN-γ, IFN-α2, IL1-β and/or IP-10, a chemokine selected from CCL2 (MCP1), CCL3, CCL5, and/or CXCL8 (IL-8), or a combination thereof.

87. The method of any one of claims 74-86, wherein the composition induces the production of an immunoglobulin in the subject.

88. The method of claim 87, wherein the immunoglobulin is specific to the antigen.

89. The method of claim 87 or claim 88, wherein the immunoglobulin is an immunoglobulin G (IgG), immunoglobulin A (IgA), or immunoglobulin M (IgM).

90. The method of claim 89, wherein the IgG is a subclass 1 IgG (IgG1) or a subclass 2 IgG (IgG2).
